SEQUENCE LISTING (4 sequences)

SEQ ID NO: 1 - protein, 450 aa, Artificial Sequence
  Polypeptide of 10D1F hIgG1
QVQLQESGPGLVKPSQTLSLTCTVSGYSITSGYSWHWIRQHPGKGLEWIGSIRYSGGTDYNPSLKSLVTISADTSKNQFSLKLSSVTAADTAVYYCARMTTAPWYPFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, Artificial Sequence
  Polypeptide of 10D1F hIgG1
DIQMTQSPSSVSASVGDRVTITCKASQIVGSNVAWYQQKPGKAPEPLIYSASYLYSGVPSRFSGSGSGTDFTLTISSLQPEDFATYYCQQYSSHPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - DNA, 1350 bp, Artificial Sequence
  Antibody heavy chain cDNA
caggtgcagctgcaggagtccggaccaggactggtgaagccatcccagaccctgtctctgacctgcacagtgtccggctactccatcacatctggctatagctggcactggatcaggcagcaccctggcaagggcctggagtggatcggcagcatccgctactccggcggcaccgactataatccctctctgaagagcctggtgaccatctccgcggacacatctaagaaccagttctccctgaagctgagctccgtgaccgccgctgatacagccgtgtactattgtgctcggatgaccacagccccctggtacccttttgactattggggccagggcaccacagtgacagtgtctagcgctagcaccaagggcccatcggtcttccccctggcaccctcctccaagagcacctctgggggcacagcggccctgggctgcctggtcaaggactacttccccgaaccggtgacggtgtcatggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccccgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 4 - DNA, 642 bp, Artificial Sequence
  Antibody light chain cDNA
gacatccagatgacccagtctcctagctccgtgagcgcctccgtgggcgatagggtgaccatcacatgcaaggccagccagatcgtgggctccaacgtggcttggtaccagcagaagcccggcaaggcccctgagccgctgatctattccgcttcatacctgtatagcggagtgccatccaggttcagcggctccggctctggaaccgactttaccctgacaatcagctccctgcagccagaggatttcgctacatattattgtcagcagtactctagccaccccctgacctttggccagggcacaaagctggagatcaagcgtacggtggcggcgccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

(19) United States
(12) Patent Application Publication
(10) Pub. No.: US 2024/0270871 A1
(43) Pub. Date: Aug. 15, 2024

(54) CELLS EXPRESSING HER3 ANTIGEN-BINDING MOLECULES

(71) Applicant: Hummingbird Bioscience Pte. Ltd., Singapore (SG)

(72) Inventors: Jerome Douglas Boyd-Kirkup, Singapore (SG); Piers Ingram, Singapore (SG); Dipti Thakkar, Singapore (SG); Siyu Guan, Singapore (SG); Akila Sadasivam, Singapore (SG)

(21) Appl. No.: 18/570,460

(22) PCT Filed: Jun. 13, 2022
(86) PCT No.: PCT/EP2022/066057

§ 371 Date: Dec. 14, 2023

(30) Foreign Application Priority Data
Jun. 14, 2021 (GB) 2108449.6

(51) Int. Cl. C07K 16/32; G01N 33/574

(52) U.S. Cl. CPC C07K 16/32; G01N 33/5748; C07K 2317/14; C07K 2317/73; C07K 2317/732; C07K 2317/76; C07K 2317/92; G01N 2333/71

(57) ABSTRACT

Cells expressing HER3 antigen-binding molecules are disclosed. Also disclosed are compositions comprising such cells, methods for producing HER3 antigen-binding molecules using such cells, and compositions comprising and methods using the HER3 antigen-binding molecules expressed by such cells.

This application claims priority from GB 2108449.6 filed 14 Jun. 2021, the contents and elements of which are herein incorporated by reference for all purposes.

### TECHNICAL FIELD

The present disclosure relates to the fields of cellular and molecular biology, and antibody technology.

### BACKGROUND

Increased HER3 expression is linked to poor prognosis in multiple solid tumors, including breast, gastric, head & neck, pancreatic, ovarian, and lung cancers. HER3-mediated signalling has adverse consequences for tumour progression; HER3 upregulation is associated with resistance to anti-HER2 and anti-EGFR therapy, and solid tumors refractory to anti-PD-1 therapy have been shown to have higher HER3 expression compared to responders to anti-PD-1 therapy.

HER3-binding antibodies are described e.g. in Zhang et al., Acta Biochimica et Biophysica Sinica (2016) 48(1): 39-48. The anti-HER3 antibody LJM-716 binds to an epitope on subdomains II and IV of the HER3 extracellular domain, locking HER3 in the inactive conformation (Garner et al., Cancer Res (2013) 73: 6024-6035). MM-121 (also known as seribantumab) has been shown to inhibit HER3-mediated signalling by blocking binding of heregulin (HRG) to HER3 (Schoeberl et al., Sci. Signal. (2009) 2(77): ra31). Patritumab (also known as U-1287 and AMG-888) also blocks binding of heregulins to HER3 (see e.g. Shimizu et al. Cancer Chemother Pharmacol. (2017) 79(3):489-495. RG7116 (also known as lumretuzumab and RO-5479599) recognises an epitope in subdomain I of the HER3 extracellular domain (see e.g. Mirschberger et al. Cancer Research (2013) 73(16) 5183-5194). KTN3379 binds to HER3 through interaction with amino acid residues in subdomain III (corresponding to the following positions of SEQ ID NO:1: Gly476, Pro477, Arg481, Gly452, Arg475, Ser450, Gly420, Ala451, Gly419, Arg421, Thr394, Leu423, Arg426, Gly427, Lys356, Leu358, Leu358, Lys356, Ala330, Lys329 and Gly337), and Met310, Glu311 and Pro328 of subdomain II (see Lee et al., Proc Natl Acad Sci USA. 2015 Oct. 27; 112(43):13225). AV-203 (also known as CAN-017) has been shown to block binding of NRG1 to HER3 and to promote HER3 degradation (see Meetze et al., Eur J Cancer 2012; 48:126). REGN1400 also inhibits binding of ligand to HER3 (see Zhang et al., Mol Cancer Ther (2014) 13:1345-1355). RG7597 (duligotuzumab) is a dual action Fab (DAF) capable of binding to both HER3 and EGFR, and binds to subdomain III of HER3 (see Schaefer et al., Cancer Cell (2011) 20(4):472-486). MM-111 and MM-141 are bispecific antibodies having HER3-binding arms which inhibit HRG ligand binding to HER3 (see McDonagh et al. Mol Cancer Ther (2012) 11:582-593 and Fitzgerald et al., Mol Cancer Ther (2014) 13:410-425).

HER3-binding antibodies and cells expressing such antibodies are described in WO 2019/185878 A1 and WO 2021/048274 A1.

### SUMMARY

In a first aspect, the present disclosure provides a cell of the cell line deposited 7 May 2021 as ATCC Patent Deposit Number PTA-127062.

The present disclosure also provides a population of cells of the cell line deposited 7 May 2021 as ATCC Patent Deposit Number PTA-127062.

The present disclosure also provides a composition comprising a cell or a population of cells according to the present disclosure.

The present disclosure also provides a method of producing an antigen-binding molecule, comprising culturing a cell or a population of cells according to the present disclosure under conditions suitable for expression of the antigen-binding molecule.

In some embodiments, the method comprises:
culturing a cell or a population of cells according to the present disclosure under conditions suitable for expression of the antigen-binding molecule; and
isolating or purifying antigen-binding molecule produced at the preceding step.

The present disclosure also provides a method of producing a pharmaceutical composition, comprising:
culturing a cell or a population of cells according to the present disclosure under conditions suitable for expression of the antigen-binding molecule; and
formulating the antigen-binding molecule produced at the preceding step with a pharmaceutically-acceptable carrier, diluent, excipient or adjuvant.

In some embodiments, the method comprises:
culturing a cell or a population of cells according to the present disclosure under conditions suitable for expression of the antigen-binding molecule;
isolating or purifying antigen-binding molecule produced at the preceding step;
formulating the isolated or purified antigen-binding molecule with a pharmaceutically-acceptable carrier, diluent, excipient or adjuvant.

The present disclosure also provides the use of a cell or a population of cells according to the present disclosure in the production of an antigen-binding molecule which binds specifically to HER3.

The present disclosure also provides the use of a cell or a population of cells according to the present disclosure in the production of pharmaceutical composition comprising an antigen-binding molecule which binds specifically to HER3.

The present disclosure also provides an antigen-binding molecule, or a plurality of antigen-binding molecules, obtained by a method according to the present disclosure.

The present disclosure also provides a pharmaceutical composition obtained by a method according to the present disclosure.

The present disclosure also provides an antigen-binding molecule, a plurality of antigen-binding molecules, or a pharmaceutical composition according to the present disclosure, for use in a method of medical treatment or prophylaxis.

The present disclosure also provides an antigen-binding molecule, a plurality of antigen-binding molecules, or a pharmaceutical composition according to the present disclosure, for use in a method of treating or preventing a cancer.

The present disclosure also provides the use of an antigen-binding molecule, a plurality of antigen-binding molecules, or a pharmaceutical composition according to the present disclosure, in the manufacture of a medicament for use in a method of treating or preventing a cancer.

The present disclosure also provides a method of treating or preventing a cancer, comprising administering to a subject a therapeutically- or prophylactically-effective amount of an antigen-binding molecule, a plurality of antigen-binding molecules, or a pharmaceutical composition according to the present disclosure.

In some embodiments in accordance with various aspects of the present disclosure, the cancer is selected from: a cancer comprising cells expressing an EGFR family member, a cancer comprising cells expressing HER3, a cancer comprising cells having a mutation resulting in increased expression of a ligand for HER3, a cancer comprising cells having an NRG gene fusion, a solid tumor, breast cancer, breast carcinoma, breast invasive carcinoma, ductal carcinoma, gastric cancer, gastric carcinoma, gastric adenocarcinoma, colorectal cancer, colorectal carcinoma, colorectal adenocarcinoma, head and neck cancer, squamous cell carcinoma of the head and neck (SCCHN), lung cancer, non-small cell lung cancer, lung adenocarcinoma, invasive mucinous lung adenocarcinoma, squamous cell lung carcinoma, ovarian cancer, ovarian carcinoma, ovarian serous adenocarcinoma, ovarian serous cystadenocarcinoma, renal cancer, renal cell carcinoma, renal clear cell carcinoma, renal cell adenocarcinoma, renal papillary cell carcinoma, pancreatic cancer, pancreatic adenocarcinoma, pancreatic ductal adenocarcinoma, cervical cancer, cervical squamous cell carcinoma, skin cancer, melanoma, esophageal cancer, esophageal adenocarcinoma, liver cancer, hepatocellular carcinoma, cholangiocarcinoma, gallbladder cancer, uterine cancer, endometrial cancer, uterine corpus endometrial carcinoma, uterine carcinosarcoma, thyroid cancer, thyroid carcinoma, pheochromocytoma, paraganglioma, bladder cancer, bladder urothelial carcinoma, prostate cancer, prostate adenocarcinoma, sarcoma, soft tissue sarcoma, thymoma, neuroendocrine tumor and neuroendocrine tumor of the nasopharynx.

The present disclosure also provides a method of inhibiting HER3-mediated signalling, comprising contacting HER3-expressing cells with an antigen-binding molecule, a plurality of antigen-binding molecules, or a pharmaceutical composition according to the present disclosure.

The present disclosure also provides a method of reducing the number or activity of HER3-expressing cells, the method comprising contacting HER3-expressing cells with an antigen-binding molecule, a plurality of antigen-binding molecules, or a pharmaceutical composition according to the present disclosure.

The present disclosure also provides an in vitro complex, optionally isolated, comprising an antigen-binding molecule according to the present disclosure bound to HER3.

The present disclosure also provides a method for detecting HER3 in a sample, comprising contacting a sample containing, or suspected to contain, HER3 with an antigen-binding molecule, a plurality of antigen-binding molecules, or a pharmaceutical composition according to the present disclosure, and detecting the formation of a complex of the antigen-binding molecule with HER3.

The present disclosure also provides a method of selecting or stratifying a subject for treatment with a HER3-targeted agent, the method comprising contacting, in vitro, a sample from the subject with an antigen-binding molecule, a plurality of antigen-binding molecules, or a pharmaceutical composition according to the present disclosure, and detecting the formation of a complex of the antigen-binding molecule with HER3.

The present disclosure also provides the use of an antigen-binding molecule, a plurality of antigen-binding molecules, or a pharmaceutical composition according to the present disclosure, as an in vitro or in vivo diagnostic or prognostic agent.

The present disclosure also provides the use of an antigen-binding molecule, a plurality of antigen-binding molecules, or a pharmaceutical composition according to the present disclosure, in a method for detecting, localizing or imaging a cancer, optionally wherein the cancer is selected from: a cancer comprising cells expressing an EGFR family member, a cancer comprising cells expressing HER3, a cancer comprising cells having a mutation resulting in increased expression of a ligand for HER3, a cancer comprising cells having an NRG gene fusion, a solid tumor, breast cancer, breast carcinoma, breast invasive carcinoma, ductal carcinoma, gastric cancer, gastric carcinoma, gastric adenocarcinoma, colorectal cancer, colorectal carcinoma, colorectal adenocarcinoma, head and neck cancer, squamous cell carcinoma of the head and neck (SCCHN), lung cancer, non-small cell lung cancer, lung adenocarcinoma, invasive mucinous lung adenocarcinoma, squamous cell lung carcinoma, ovarian cancer, ovarian carcinoma, ovarian serous adenocarcinoma, ovarian serous cystadenocarcinoma, renal cancer, renal cell carcinoma, renal clear cell carcinoma, renal cell adenocarcinoma, renal papillary cell carcinoma, pancreatic cancer, pancreatic adenocarcinoma, pancreatic ductal adenocarcinoma, cervical cancer, cervical squamous cell carcinoma, skin cancer, melanoma, esophageal cancer, esophageal adenocarcinoma, liver cancer, hepatocellular carcinoma, cholangiocarcinoma, gallbladder cancer, uterine cancer, endometrial cancer, uterine corpus endometrial carcinoma, uterine carcinosarcoma, thyroid cancer, thyroid carcinoma, pheochromocytoma, paraganglioma, bladder cancer, bladder urothelial carcinoma, prostate cancer, prostate adenocarcinoma, sarcoma, soft tissue sarcoma, thymoma, neuroendocrine tumor and neuroendocrine tumor of the nasopharynx.

The present disclosure also provides a kit of parts, comprising: a cell, a population of cells, a composition, an antigen-binding molecule, a plurality of antigen-binding molecules or a pharmaceutical composition according to the present disclosure.

### DESCRIPTION

The present disclosure relates to a cell line having particularly advantageous properties, which expresses a HER3-binding antibody.

### Cells

The present disclosure provides the cell line deposited 7 May 2021 as ATCC Patent Deposit Number PTA-127062. Also provided are cells of the cell line deposited 7 May 2021 as ATCC Patent Deposit Number PTA-127062, and populations of such cells.

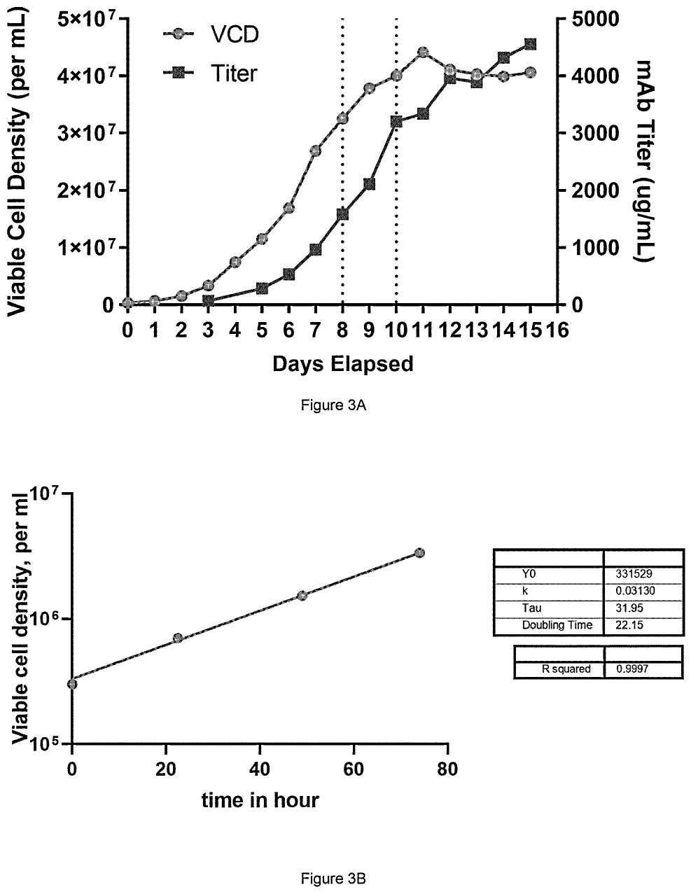

Production of the cell line deposited 7 May 2021 as ATCC Patent Deposit Number PTA-127062 is described in Example 2 herein. The cell line was prepared from cells of the CHO-k1 cell line (ATCC, Cat. No. CCL-61), by electroporation with the polycistronic vector represented schematically in FIG. 3, encoding: (i) 10D1F hIgG1 heavy chain, having the amino acid sequence shown in SEQ ID NO:1, and (ii) 10D1F K light chain, having the amino acid sequence shown in SEQ ID NO:2.

Example 3 describes characterisation of the cell line deposited 7 May 2021 as ATCC Patent Deposit Number PTA-127062. The cell line deposited 7 May 2021 as ATCC Patent Deposit Number PTA-127062 is provided with the following advantageous properties:
High overall productivity, producing secreted 10D1 F hIgG1 at an antibody titer >4.3 g/L in culture;
High cell-specific productivity (pg/cell/day);
High viability in culture;
High growth rate, having a doubling time <24 hours;
Ability to grow to high density in culture, having a peak cell density >40×10⁶ cells/ml;
Ability to consume lactate via a lactate consumption metabolic pathway, thereby reducing lactate accumulation in culture;
Produces 10D1F hIgG1 having very low aggregation propensity (and high purity of the correctly-assembled antibody species);
Produces 10D1 F hIgG1 retaining binding to human HER3 with sub-picomolar affinity; and High phenotypic stability, maintaining high antibody titer (>4.3 g/L), high cell-specific productivity, high viability in culture, and production of 10D1F hIgG1 having very low aggregation propensity (and high purity of the correctly-assembled species antibody species), and retaining binding to human HER3 with sub-picomolar affinity for at least 88 generations.

The cell line deposited 7 May 2021 as ATCC Patent Deposit Number PTA-127062 is demonstrated in the present examples to possess a superior overall profile of characteristics compared to other 10D1 F hIgG1-producing cell lines.

### Methods for Producing Antigen-Binding Molecules and Compositions

The present disclosure provides a methods for producing an antigen-binding molecule, comprising culturing a cell or a population of cells of the cell line deposited 7 May 2021 as ATCC Patent Deposit Number PTA-127062 under conditions suitable for expression of the antigen-binding molecule.

It will be appreciated that the cell line deposited 7 May 2021 as ATCC Patent Deposit Number PTA-127062 comprises nucleic acid encoding the constituent polypeptides of 10D1F hIgG1 (i.e. the polypeptides of SEQ ID NO:1 and SEQ ID NO:2). Thus, the antigen-binding molecule produced by culturing a cell or a population of cells of the cell line deposited 7 May 2021 as ATCC Patent Deposit Number PTA-127062 under conditions suitable for expression of the antigen-binding molecule is 10D1F hIgG1.

Suitable culture conditions for the expression of 10D1F hIgG1 from cells of the cell line deposited 7 May 2021 as ATCC Patent Deposit Number PTA-127062 will be apparent to person skilled in the art. Culture conditions for the expression of monoclonal antibodies from mammalian cells in culture are described e.g. in Birch and Racher, Adv Drug Deliv Rev (2006) 58(5-6):671-85 and Li et al., MAbs (2010) 2(5): 466-477, both of which are hereby incorporated by reference in its entirety. Suitable culture conditions include conditions suitable for the maintenance of cells of the CHO-k1 cell line (ATCC, Cat. No. CCL-61) in in vitro culture.

In some embodiments the culture is performed in culture medium comprising EX-CELL Advanced CHO Fed-Batch Medium (SAFC) or F-12K Medium (ATCC).

In some embodiments, the culture is performed in cell culture medium comprising added L-Glutamine. In some embodiments, the culture is performed in cell culture medium comprising 2 to 12 mM L-Glutamine, e.g. 4 to 10 mM L-Glutamine, e.g. ˜6 mM L-Glutamine.

In some embodiments, the culture is performed in cell culture medium comprising added methotrexate. In some embodiments, the culture is performed in cell culture medium comprising 200 to 300 mM methotrexate, e.g. 225 to 275 mM methotrexate, e.g. ˜250 mM methotrexate. In some embodiments, the culture is performed in the absence of methotrexate.

In some embodiments, the culture is performed in the absence of fetal bovine serum (FBS). In some embodiments, the culture is performed in the absence of human serum. In some embodiments, the culture is performed in the absence of serum. That is, in some embodiments the cells are cultured under ‘serum-free’ conditions.

In particular embodiments, the culture is performed in culture medium comprising EX-CELL Advanced CHO Fed-Batch Medium (SAFC), 2 to 12 mM L-Glutamine (e.g. 4 to 10 mM L-Glutamine, e.g. ˜6 mM L-Glutamine), 200 to 300 mM methotrexate (e.g. 225 to 275 mM methotrexate, e.g. ˜250 mM methotrexate), and in the absence of serum.

It will be appreciated that the culture is performed under suitable environmental conditions, e.g. at 37° C., in 4-10% CO₂ (e.g. 5-8% CO₂), and in a humidified incubator (e.g. at 95% humidity), with or without agitation (e.g. agitation at 75 to 175 rpm, e.g. 90 to 130 rpm, e.g. ˜110 rpm). Culture may be performed in a bioreactor provided with an appropriate supply of nutrients, air/oxygen and/or growth factors.

Bioreactors include one or more vessels in which cells may be cultured. Culture in the bioreactor may occur continuously, with a continuous flow of reactants into, and a continuous flow of cultured cells from, the reactor. Alternatively, the culture may occur in batches. The bioreactor monitors and controls environmental conditions such as pH, oxygen, flow rates into and out of, and agitation within the vessel such that optimum conditions are provided for the cells being cultured.

In some embodiments the methods comprise isolating or purifying antigen-binding molecule produced by culturing a cell or a population of cells of the cell line deposited 7 May 2021 as ATCC Patent Deposit Number PTA-127062 under conditions suitable for expression of the antigen-binding molecule.

Following culturing the cells that express the antigen-binding molecules, the expressed antigen-binding molecules may be isolated. Secreted antigen-binding molecules can be collected by partitioning culture media from the cells (e.g. by centrifugation), and isolating/purifying the secreted antigen-binding molecules from the culture media.

Techniques for the isolation/purification of antigen-binding molecules from compositions comprising heterogeneous populations of proteins are well known to those of skill in the art, and are described, for example, in Green and Sambrook, Molecular Cloning: A Laboratory Manual (4th Edition), Birch and Racher, Adv Drug Deliv Rev (2006) 58(5-6):671-85, and Murphy et al., Antibody Technology Journal (2016) 6: 17-32, all of which are hereby incorporated by reference in their entirety.

Large-scale purification of antigen-binding molecules is commonly based on affinity chromatography, and Protein A or G affinity purification is used in many cases. Purification may alternatively, or additionally, comprise purification by anion/cation exchange chromatography, hydrophobic interaction chromatography and/or size exclusion chromatography, which are well known to the person skilled in the art.

The various purification steps are designed to remove contaminant proteins from the cells or culture media to ppm levels, and to reduce DNA to ppb levels. Depending on the processes used, there may be additional specific contaminants to be removed (e.g. leached protein A/G). Purification may comprise filtration (e.g. using a 0.22 μm filter) to remove potential biological contaminants. In addition to contaminants, it may also be necessary to remove undesirable derivatives of the antigen-binding molecule, such as aggregates and degradation products.

In some embodiments, isolating or purifying antigen-binding molecule according to the present disclosure comprises isolation/purification by one or more of: affinity chromatography, (e.g. Protein G chromatography or Protein A chromatography), size exclusion chromatography, high-performance liquid chromatography, ultra-performance liquid chromatography, and ion-exchange chromatography.

Following isolation/purification, the antigen-binding molecule may be provided in a suitable buffer, e.g. for storage. As used herein, a “buffer” refers to a buffered solution that resists changes in pH by the action of its acid-base conjugate components. A composition comprising an antigen-binding molecule according to the present disclosure may comprise the antigen-binding molecule in a buffer.

A buffer of the present disclosure preferably has a pH in the range from about 4.5 to about 7.0, preferably from about 5.0 to about 6.5. Examples of buffers that will control the pH in this range include acetate, histidine, histidine-arginine, histidine-methionine and other organic acid buffers. Antigen-binding molecules may be buffer exchanged into a buffer of interest by buffer dialysis.

In some embodiments, methods of the present disclosure comprise formulating an antigen-binding molecule according to the present disclosure to a composition, e.g. a pharmaceutical composition. In some embodiments the methods comprise mixing an antigen-binding molecule, or mixing a composition comprising an antigen-binding molecule, with a pharmaceutically-acceptable carrier, diluent, excipient or adjuvant.

The present disclosure also provides antigen-binding molecules and compositions produced by the methods described herein.

### Compositions

The present disclosure also provides a composition comprising a cell, or a population of cells, of the cell line deposited 7 May 2021 as ATCC Patent Deposit Number PTA-127062.

The present disclosure also provides an antigen-binding molecule expressed from a cell, or a population of cells, of the cell line deposited 7 May 2021 as ATCC Patent Deposit Number PTA-127062. The present disclosure also provides a composition comprising an antigen-binding molecule according to the present disclosure.

Compositions may comprise an antigen-binding molecule according to the present disclosure provided in a buffer, e.g. a buffer as described herein.

Antigen-binding molecules described herein may be formulated as pharmaceutical compositions or medicaments for clinical use, and may comprise a pharmaceutically-acceptable carrier, diluent, excipient or adjuvant.

Compositions according to the present disclosure may be formulated for topical, parenteral, systemic, intracavitary, intravenous, intra-arterial, intramuscular, intrathecal, intraocular, intraconjunctival, intratumoral, subcutaneous, intradermal, intrathecal, oral or transdermal routes of administration which may include injection or infusion. Such compositions may comprise the antigen-binding molecule or cell in a sterile or isotonic medium. Medicaments and pharmaceutical compositions may be formulated in fluid, including gel, form. Fluid formulations may be formulated for administration by injection or infusion (e.g. via catheter) to a selected region of the human or animal body. In some embodiments, compositions of the present disclosure may be formulated for injection or infusion, e.g. into a blood vessel or tumor.

### Therapeutic and Prophylactic Applications

The compositions comprising cells of the cell line deposited 7 May 2021 as ATCC Patent Deposit Number PTA-127062, antigen-binding molecules produced from such cells and compositions comprising such antigen-binding molecules find use in therapeutic and prophylactic methods. The therapeutic and prophylactic utility of 10D1F hIgG1 is evidenced by WO 2019/185878 A1 and WO 2021/048274 A1 (which are incorporated by reference in their entirety).

The present disclosure provides an antigen-binding molecule or composition according to the present disclosure for use in a method of medical treatment or prophylaxis. Also provided is the use of an antigen-binding molecule or composition according to the present disclosure in the manufacture of a medicament for treating or preventing a disease or condition. Also provided is a method of treating or preventing a disease or condition, comprising administering to a subject a therapeutically or prophylactically effective amount of an antigen-binding molecule or composition described herein.

The methods may be effective to reduce the development or progression of a disease/condition, alleviate the symptoms of a disease/condition or reduce the pathology of a disease/condition. The methods may be effective to prevent progression of the disease/condition, e.g. to prevent worsening of, or to slow the rate of development of, the disease/condition. In some embodiments the methods may lead to an improvement in the disease/condition, e.g. a reduction in the symptoms of the disease/condition or reduction in some other correlate of the severity/activity of the disease/condition. In some embodiments the methods may prevent development of the disease/condition to a later stage (e.g. a chronic stage or metastasis).

It will be appreciated that the antigen-binding molecules and compositions described herein may be used for the treatment/prevention of any disease/condition that would derive therapeutic or prophylactic benefit from a reduction in the number and/or activity of cells expressing HER3. For example, the disease/condition may be a disease/condition in which cells expressing HER3 are pathologically implicated, e.g. a disease/condition in which an increased number/proportion of cells expressing HER3 is positively associated with the onset, development or progression of the disease/condition, and/or severity of one or more symptoms of the disease/condition, or for which an increased number/proportion of cells expressing HER3, is a risk factor for the onset, development or progression of the disease/condition.

In some embodiments, the disease/condition to be treated/prevented in accordance with the present disclosure is a disease/condition characterised by an increase in the number/proportion/activity of cells expressing HER3, e.g. as compared to the number/proportion/activity of cells expressing HER3 in the absence of the disease/condition.

In some embodiments the disease/condition to be treated/prevented is a cancer.

The cancer may be any unwanted cell proliferation (or any disease manifesting itself by unwanted cell proliferation), neoplasm or tumor. The cancer may be benign or malignant and may be primary or secondary (metastatic). A neoplasm or tumor may be any abnormal growth or proliferation of cells and may be located in any tissue. The cancer may be of tissues/cells derived from e.g. the adrenal gland, adrenal medulla, anus, appendix, bladder, blood, bone, bone marrow, brain, breast, cecum, central nervous system (including or excluding the brain) cerebellum, cervix, colon, duodenum, endometrium, epithelial cells (e.g. renal epithelia), gallbladder, oesophagus, glial cells, heart, ileum, jejunum, kidney, lacrimal glad, larynx, liver, lung, lymph, lymph node, lymphoblast, maxilla, mediastinum, mesentery, myometrium, nasopharynx, omentum, oral cavity, ovary, pancreas, parotid gland, peripheral nervous system, peritoneum, pleura, prostate, salivary gland, sigmoid colon, skin, small intestine, soft tissues, spleen, stomach, testis, thymus, thyroid gland, tongue, tonsil, trachea, uterus, vulva, and/or white blood cells.

Tumors to be treated may be nervous or non-nervous system tumors. Nervous system tumors may originate either in the central or peripheral nervous system, e.g. glioma, medulloblastoma, meningioma, neurofibroma, ependymoma, Schwannoma, neurofibrosarcoma, astrocytoma and oligodendroglioma. Non-nervous system cancers/tumors may originate in any other non-nervous tissue, examples include melanoma, mesothelioma, lymphoma, myeloma, leukemia, Non-Hodgkin's lymphoma (NHL), Hodgkin's lymphoma, chronic myelogenous leukemia (CML), acute myeloid leukemia (AML), myelodysplastic syndrome (MDS), cutaneous T-cell lymphoma (CTCL), chronic lymphocytic leukemia (CLL), hepatoma, epidermoid carcinoma, prostate carcinoma, breast cancer, lung cancer, colon cancer, ovarian cancer, pancreatic cancer, thymic carcinoma, NSCLC, hematologic cancer and sarcoma.

HER3 and its association with and role in cancer is reviewed e.g. in Karachaliou et al., BioDrugs. (2017) 31(1):63-73 and Zhang et al., Acta Biochimica et Biophysica Sinica (2016) 48(1): 39-48, both of which are hereby incorporated by reference in their entirety.

In some embodiments, a cancer is selected from: a cancer comprising cells expressing an EGFR family member, a cancer comprising cells expressing HER3, a cancer comprising cells having a mutation resulting in increased expression of a ligand for HER3, a cancer comprising cells having an NRG gene fusion, a solid tumor, breast cancer, breast carcinoma, breast invasive carcinoma, ductal carcinoma, gastric cancer, gastric carcinoma, gastric adenocarcinoma, colorectal cancer, colorectal carcinoma, colorectal adenocarcinoma, head and neck cancer, squamous cell carcinoma of the head and neck (SCCHN), lung cancer, non-small cell lung cancer, lung adenocarcinoma, invasive mucinous lung adenocarcinoma, squamous cell lung carcinoma, ovarian cancer, ovarian carcinoma, ovarian serous adenocarcinoma, ovarian serous cystadenocarcinoma, renal cancer, renal cell carcinoma, renal clear cell carcinoma, renal cell adenocarcinoma, renal papillary cell carcinoma, pancreatic cancer, pancreatic adenocarcinoma, pancreatic ductal adenocarcinoma, cervical cancer, cervical squamous cell carcinoma, skin cancer, melanoma, esophageal cancer, esophageal adenocarcinoma, liver cancer, hepatocellular carcinoma, cholangiocarcinoma, gallbladder cancer, uterine cancer, endometrial cancer, uterine corpus endometrial carcinoma, uterine carcinosarcoma, thyroid cancer, thyroid carcinoma, pheochromocytoma, paraganglioma, bladder cancer, bladder urothelial carcinoma, prostate cancer, prostate adenocarcinoma, sarcoma, soft tissue sarcoma, thymoma, neuroendocrine tumor and neuroendocrine tumor of the nasopharynx.

In some embodiments the cancer to be treated in accordance with the present invention is selected from: a HER3-expressing cancer, gastric cancer (e.g. gastric carcinoma, gastric adenocarcinoma, gastrointestinal adenocarcinoma), head and neck cancer (e.g. head and neck squamous cell carcinoma), breast cancer, ovarian cancer (e.g. ovarian carcinoma), lung cancer (e.g. NSCLC, lung adenocarcinoma, squamous lung cell carcinoma), melanoma, prostate cancer, oral cavity cancer (e.g. oropharyngeal cancer), renal cancer (e.g. renal cell carcinoma) or colorectal cancer (e.g. colorectal carcinoma), oesophageal cancer, pancreatic cancer, a solid cancer and/or a liquid cancer.

The treatment/prevention may be aimed at one or more of: delaying/preventing the onset/progression of symptoms of the cancer, reducing the severity of symptoms of the cancer, reducing the survival/growth/invasion/metastasis of cells of the cancer, reducing the number of cells of the cancer and/or increasing survival of the subject.

In some embodiments, the cancer to be treated/prevented comprises cells expressing an EGFR family member (e.g. HER3, EGFR, HER2 or HER4), and/or cells expressing a ligand for an EGFR family member. In some embodiments, the cancer to be treated/prevented is a cancer which is positive for an EGFR family member. In some embodiments, the cancer over-expresses an EGFR family member and/or a ligand for an EGFR family member. Overexpression can be determined by detection of a level of expression which is greater than the level of expression by equivalent non-cancerous cells/non-tumor tissue.

Expression may be determined by any suitable means. Expression may be gene expression or protein expression. Gene expression can be determined e.g. by detection of mRNA encoding HER3, for example by quantitative real-time PCR (qRT-PCR). Protein expression can be determined e.g. by for example by antibody-based methods, for example by western blot, immunohistochemistry, immunocytochemistry, flow cytometry, or ELISA.

In some embodiments, the cancer to be treated/prevented comprises cells expressing HER3. In some embodiments, the cancer to be treated/prevented is a cancer which is positive for HER3. In some embodiments, the cancer over-expresses HER3. Overexpression of HER3 can be determined by detection of a level of expression of HER3 which is greater than the level of expression by equivalent non-cancerous cells/non-tumor tissue.

In some embodiments, a patient may be selected for treatment described herein based on the detection of a cancer expressing HER3, or overexpressing HER3, e.g. in a sample obtained from the subject.

In some embodiments, the cancer to be treated/prevented comprises cells expressing a ligand for HER3 (e.g. NRG1 and/or NRG2). In some embodiments, the cancer to be treated/prevented comprises cells expressing a level of expression of NRG1 and/or NRG2 which is greater than the level of expression by equivalent non-cancerous cells/non-tumor tissue.

HER3-binding antigen-binding molecules described herein bind to HER3 with extremely high affinity when HER3 is bound by NRG (i.e. when HER3 is provided in the ‘open’ conformation), and also when HER3 is not bound by NRG (i.e. when HER3 is provided in the ‘closed’ conformation).

Thus, the antigen-binding molecules and compositions of the present disclosure are particularly useful for the treatment/prevention of cancers characterised by HER3 ligand expression/overexpression, for example cancers/tumors comprising cells expressing/overexpressing a ligand for HER3.

In some embodiments, the cancer to be treated in accordance with the present disclosure comprises cells harbouring a genetic variant (e.g. a mutation) which causes increased (gene and/or protein) expression of a ligand for HER3, relative to comparable cells harbouring a reference allele not comprising the genetic variant (e.g. a non-mutated, or ‘wildtype’ allele). The genetic variant may be or comprise insertion, deletion, substitution to, or larger-scale translocation/rearrangement of, the nucleotide sequence relative to the reference allele.

A mutation ‘resulting in’ increased expression of a ligand for HER3 may be known or predicted to cause, or may be associated with, increased gene/protein expression of a ligand for HER3. Mutations resulting in increased expression of a ligand for HER3 may be referred to as ‘activating’ mutations.

A mutation which causes increased expression of a ligand for HER3 may result in gene or protein expression of a ligand for HER3 which is not expressed by, and/or not encoded by genomic nucleic acid of, an equivalent cell not harbouring the mutation. That is, the ligand for HER3 may be a neoantigen arising as a result of the mutation, and thus ‘increased expression’ may be from zero expression. By way of illustration, a cell comprising CD47-NRG1 gene fusion displays increased expression of the CD47-NRG1 fusion polypeptide encoded by the gene fusion relative to cells lacking the CD47-NRG1 gene fusion.

A mutation which causes increased expression of a ligand for HER3 may result in increased gene or protein expression of a ligand for HER3 which is expressed by, and/or which is encoded by genomic nucleic acid of, an equivalent cell not comprising the mutation. By way of illustration, a cell may comprise a mutation resulting in an increase in the level of transcription of nucleic acid encoding NRG1 relative to level of transcription of nucleic acid encoding NRG1 by an equivalent cell not comprising the mutation.

In some embodiments, a mutation which causes increased expression of a ligand for HER3 may cause an increase in gene expression of a ligand for HER3 relative to an equivalent cell not comprising the mutation. In some embodiments, a mutation which causes increased expression of a ligand for HER3 may cause an increase in protein expression of a ligand for HER3 relative to an equivalent cell not comprising the mutation.

In some embodiments, a mutation which causes increased expression of a ligand for HER3 may cause an increase in the level of a ligand for HER3 on or at the cell surface of a cell comprising the mutation, relative to an equivalent cell not comprising the mutation. In some embodiments, a mutation which causes increased expression of a ligand for HER3 may cause an increase in the level of a secretion of a ligand for HER3 from a cell comprising the mutation, relative to an equivalent cell not comprising the mutation.

Cells having increased expression of a ligand for HER3 relative to the level of expression of the ligand for HER3 by a reference cell (e.g. as a result of mutation) may be described as ‘overexpressing’ the ligand for HER3, or having ‘upregulated expression’ of the ligand for HER3. For example, a cancer comprising cells harbouring a mutation resulting in increased expression of a ligand for HER3 relative to equivalent cells lacking the mutation may be described as a cancer comprising cells displaying overexpression or upregulated expression of the ligand for HER3. In some embodiments, the reference cell lacking the mutation may be a non-cancerous cell (e.g. of equivalent cell type) or a cancerous cell (e.g. of equivalent cancer type).

Herein, a ‘ligand for HER3’ is generally intended to refer to molecule capable of binding to HER3 through the ligand binding region of HER3 formed by domains I and III of HER3. In some embodiments, a ligand for HER3 binds to HER3 via interaction with domains I and/or III of HER3. Exemplary ligands for HER3 include Neuregulins such as NRG1 and NRG2, which bind to HER3 via interaction between their EGF-like domains and the ligand binding region of HER3.

The HER3 ligand is preferably able to bind and trigger signalling through the HER3 receptor and/or receptor complexes comprising HER3. As will be clear from the present disclosure, receptor complexes comprising HER3 may further comprise an interaction partner for HER3 as described herein, e.g. HER3, HER2, EGFR, HER4, HGFR, IGF1R and/or cMet).

In some embodiments the ligand for HER3 is able to bind to HER3 receptor/receptor complex expressed by a cell other than the cell having increased expression of the HER3 ligand. For example, in some embodiments the ligand for HER3 is able to bind to a HER3-expressing cancer cell.

In some embodiments the ligand for HER3 is able to bind to HER3 receptor/receptor complex expressed by the cell having increased expression of the HER3 ligand.

In some embodiments the cancer to be treated comprises (i) cells expressing HER3, and (ii) cells expressing a ligand for HER3 (e.g. having increased expression of a ligand for HER3, e.g. as a consequence of mutation resulting in increased expression of a ligand for HER3).

In some embodiments the cancer to be treated comprises cells which (i) express HER3 and (ii) which also express a ligand for HER3 (e.g. which have increased expression of a ligand for HER3, e.g. as a consequence of mutation resulting in increased expression of a ligand for HER3).

In some embodiments, the ligand for HER3 comprises, or consists of, the amino acid sequence a HER3-binding region of a ligand for HER3, or an amino acid sequence derived from a HER3-binding region of a ligand for HER3. An amino acid sequence which is derived from a HER3-binding region of a ligand for HER3 may comprise at least 60% (e.g. 70%, 75%, 80%, 85%, 90%, 91%, 92%, 93%, 94%, 95%, 96%, 97%, 98%, 99% or 100%) amino acid sequence identity to the amino acid sequence from which it is derived.

In some embodiments, the ligand for HER3 comprises an EGF-like domain capable of binding to HER3, or a HER3-binding fragment thereof. In some embodiments, a HER3-binding EGF-like domain/fragment is, or is derived from, an EGF family member (e.g. heparin-binding EGF-like growth factor (HB-EGF), transforming growth factor-α (TGF-α), amphiregulin (AR), epiregulin (EPR), epigen, betacellulin (BTC), NRG1, NRG2, NRG3 or NRG4).

EGF family members contain one or more repeats of the conserved amino acid sequence shown in SEQ ID NO:240 of WO 2021/048274 A1, which contains six cysteine residues that form three intramolecular disulfide bonds, providing three structural loops required for high-affinity binding to their cognate receptors (see Harris et al. Experimental Cell Research (2003) 284(1): 2-13). In some embodiments, a ligand for HER3 comprises one or more copies of an amino acid sequence conforming to the consensus sequence shown in SEQ ID NO:240 of WO 2021/048274 A1.

Exemplary ligands for HER3 include Neuregulins (NRGs). Neuregulins include NRG1, NRG2, NRG3 and NRG4. The amino acid sequence of human NRG1 (alpha isoform) is shown in SEQ ID NO:232 of WO 2021/048274 A1. The alpha isoform and several other isoforms of human NRG1 (including alpha1a isoform (see UnitProt: Q02297-2), alpha2b isoform (see UnitProt: Q02297-3) and alpha3 isoform (see UnitProt: Q02297-4)) comprise the EGF-like domain shown in SEQ ID NO:233 of WO 2021/048274 A1, through which they bind to HER3. The amino acid sequence of human NRG2 (isoform 1) is shown in SEQ ID NO:234 of WO 2021/048274 A1. Isoform 1 and several other isoforms of human NRG2 (including isoform 3 (see UniProt:014511-3), isoform 5 (see UniProt:014511-5), isoform 6 (see UniProt:014511-6), isoform DON-1B (see UniProt:014511-7) and isoform DON-1R (see UniProt:014511-8)) comprise the EGF-like domain shown in SEQ ID NO:235 of WO 2021/048274 A1, through which they bind to HER3. The amino acid sequence of human NRG3 is shown in SEQ ID NO:236 of WO 2021/048274 A1, and the EGF-like domain of human NRG3 shown in SEQ ID NO:237 of WO 2021/048274 A1. The amino acid sequence of human NRG4 is shown in SEQ ID NO:238 of WO 2021/048274 A1, and the EGF-like domain of human NRG3 shown in SEQ ID NO:239 of WO 2021/048274 A1. In some embodiments, an NRG is selected from NRG1, NRG2, NRG3 and NRG4. In some embodiments, an NRG is selected from NRG1 and NRG2.

In some embodiments an EGF-like domain/fragment comprises, or consists of, an amino acid sequence having at least 60% (e.g. 70%, 75%, 80%, 85%, 90%, 91%, 92%, 93%, 94%, 95%, 96%, 97%, 98%, 99% or 100%) amino acid sequence identity to the EGF-like domain of an NRG (NRG1, NRG2, NRG3 or NRG4). In some embodiments an EGF-like domain/fragment comprises, or consists of, an amino acid sequence having at least 60% (e.g. 70%, 75%, 80%, 85%, 90%, 91%, 92%, 93%, 94%, 95%, 96%, 97%, 98%, 99% or 100%) amino acid sequence identity to one of SEQ ID NOs:233, 235, 237 or 239 of WO 2021/048274 A1.

In some embodiments, the ligand for HER3 is an NRG (e.g. NRG1, NRG2, NRG3 or NRG4; e.g. NRG1 or NRG2), or comprises an amino acid sequence derived from an amino acid sequence of an NRG (i.e. comprises an amino acid sequence having at least 60% (e.g. 70%, 75%, 80%, 85%, 90%, 91%, 92%, 93%, 94%, 95%, 96%, 97%, 98%, 99% or 100%) amino acid sequence identity to an amino acid sequence of an NRG.

In some embodiments, the ligand for HER3 comprises, or consists of, an amino acid sequence having at least 60% (e.g. 70%, 75%, 80%, 85%, 90%, 91%, 92%, 93%, 94%, 95%, 96%, 97%, 98%, 99% or 100%) amino acid sequence identity to the HER3-binding region of a ligand for HER3 (e.g. an NRG, e.g. NRG1, NRG2, NRG3 or NRG4; e.g. NRG1 or NRG2). In some embodiments, a ligand for HER3 comprises, or consists of, an amino acid sequence having at least 60% (e.g. 70%, 75%, 80%, 85%, 90%, 91%, 92%, 93%, 94%, 95%, 96%, 97%, 98%, 99% or 100%) amino acid sequence identity to the EGF-like domain of an NRG (e.g. NRG1, NRG2, NRG3 or NRG4; e.g. NRG1 or NRG2).

In some embodiments a ligand for HER3 is not an EGFR family protein (e.g. HER3, HER2, EGFR, HER4, HGFR, IGF1R, cMet).

In some embodiments, the mutation resulting in increased expression of a ligand for HER3 is an NRG gene fusion. In some embodiments, the ligand for HER3 is the product of (i.e. a polypeptide encoded by) an NRG gene fusion. In some embodiments the cancer comprises cells having an NRG gene fusion.

As used herein, an “NRG gene fusion” refers to a genetic variant encoding a polypeptide comprising (i) an amino acid sequence of an NRG protein (e.g. NRG1, NRG2, NRG3 or NRG4; e.g. NRG1 or NRG2), and (ii) an amino acid sequence of a protein other than the NRG protein.

It will be appreciated that an NRG gene fusion preferably encodes a HER3 ligand as described herein. In some embodiments, an NRG gene fusion encodes a polypeptide comprising a HER3-binding region of an NRG protein. In some embodiments, an NRG gene fusion encodes a polypeptide comprising the EGF-like domain of an NRG protein, or an amino acid sequence which is capable of binding to HER3 and having at least 60% (e.g. 70%, 75%, 80%, 85%, 90%, 91%, 92%, 93%, 94%, 95%, 96%, 97%, 98%, 99% or 100%) amino acid sequence identity to the EGF-like domain of an NRG protein.

In some embodiments, an NRG gene fusion encodes a fusion polypeptide comprising a transmembrane domain. In some embodiments, an NRG gene fusion encodes a fusion polypeptide comprising the transmembrane domain of a protein other than the NRG protein.

In some embodiments, an NRG gene fusion is an NRG1 gene fusion. In some embodiments, the NRG1 gene fusion encodes a polypeptide comprising the EGF-like domain of NRG1, or an amino acid sequence which is capable of binding to HER3 and having at least 60% (e.g. 70%, 75%, 80%, 85%, 90%, 91%, 92%, 93%, 94%, 95%, 96%, 97%, 98%, 99% or 100%) amino acid sequence identity to the EGF-like domain of NRG1.

NRG1 gene fusions are described e.g. in WO 2018/182422 A1, WO 2019/051155 A1, Dhanasekaran et al., Nat Commun. (2014) 5: 5893, Drilon et al., Cancer Discov. (2018) 8(6):686-695, Nagasaka et al., Journal of Thoracic Oncology (2019) 14(8):1354-1359 and Jonna et al., Clin Cancer Res. (2019) 25(16):4966-4972, all of which are hereby incorporated by reference in their entirety. The diversity of NRG1 gene fusions may result from NRG1 being located on chromosome 8, which is particularly susceptible to genomic translocation events (Adelaide et al., Genes Chromosomes Cancer. (2003) 37(4):333-45).

In some embodiments, an NRG1 gene fusion is selected from CLU-NRG1, CD74-NRG1, DOC4-NRG1, SLC3A2-NRG1, RBPMS-NRG1, WRN-NRG1, SDC4-NRG1, RAB21L1-NRG1, VAMP2-NRG1, KIF13B-NRG1, THAP7-NRG1, SMAD4-NRG1, MDK-NRG1, TNC-NRG1, DIP2B-NRG1, MRPL13-NRG1, PARP8-NRG1, ROCK1-NRG1, DPYSL2-NRG1, ATP1B1-NRG1, CDH6-NRG1, APP-NRG1, AKAP13-NRG1, THBS1-NRG1, FOXA1-NRG1, PDE7A-NRG1, RAB31L1-NRG1, CDK1-NRG1, BMPRIB-NRG1, TNFRSF10B-NRG1, and MCPH1-NRG1. In some embodiments, an NRG1 gene fusion is CLU-NRG1.

CD74-NRG1 gene fusion is described e.g. in Fernandez-Cuesta et al. Cancer Discov. (2014) 4:415-22 and Nakaoku et al., Clin Cancer Res (2014) 20:3087-93. DOC4-NRG1 gene fusion is described e.g. in Liu et al., Oncogene. (1999) 18(50):7110-4 and Wang et al., Oncogene. (1999) 18(41):5718-21. SLC3A2-NRG1 gene fusion is described e.g. in Nakaoku et al., Clin Cancer Res (2014) 20:3087-93, Shin et al., Oncotarget (2016) 7:69450-65 and Shin et al., Mol Cancer Ther. (2018) 17(9):2024-2033. RBPMS-NRG1, WRN-NRG1, RAB21L1-NRG1 and SDC4-NRG1 gene fusions are described e.g. in Dhanasekaran et al., Nat Commun. (2014) 5: 5893. VAMP2-NRG1 gene fusion is described e.g. in Jung et al., J Thorac Oncol. (2015) 10(7):1107-11 and Shim et al., J Thorac Oncol. (2015) 10(8):1156-62. KIF13B-NRG1 gene fusion is described e.g. in Xia et al., Int J Surg Pathol. (2017) 25(3):238-240. SMAD4-NRG1, AKAP13-NRG1, THBS1-NRG1, FOXA1-NRG1, PDE7A-NRG1, RAB31L1-NRG1 and THAP7-NRG1 gene fusions are described e.g. in Drilon et al., Cancer Discov. (2018) 8(6):686-695. MDK-NRG1, TNC-NRG1, DIP2B-NRG1, MRPL13-NRG1, PARP8-NRG1, ROCK1-NRG1 and DPYSL2-NRG1 gene fusions are described e.g. in Jonna et al., Clin Cancer Res. (2019) 25(16):4966-4972. ATP1B1-NRG1 gene fusion is described e.g. in Drilon et al., Cancer Discov. (2018) 8(6):686-695 and Jones et al., Annals of Oncology (2017) 28:3092-3097. CLU-NRG1 gene fusion is described e.g. in Drilon et al., Cancer Discov. (2018) 8(6):686-695 and Nagasaka et al., Journal of Thoracic Oncology (2019) 14(8):1354-1359.

In some embodiments, an NRG gene fusion is an NRG2 gene fusion. In some embodiments, the NRG2 gene fusion encodes a polypeptide comprising the EGF-like domain of NRG2, or an amino acid sequence which is capable of binding to HER3 and having at least 60% (e.g. 70%, 75%, 80%, 85%, 90%, 91%, 92%, 93%, 94%, 95%, 96%, 97%, 98%, 99% or 100%) amino acid sequence identity to the EGF-like domain of NRG2. NRG2 gene fusions include SLC12A2-NRG2 described e.g. in WO 2015/093557 A1, and ZNF208-NRG2 described in Dupain et al., Mol Ther. (2019) 27(1):200-218.

A cancer comprising cells having a mutation which results in increased expression of a ligand for HER3 (e.g. comprising cells having an NRG gene fusion, e.g. an NRG1 gene fusion or an NRG2 gene fusion) can be any cancer described herein. In some embodiments, such cancer may be of tissues/cells derived from the lung, breast, head, neck, kidney, ovary, pancreas, prostate, uterus, gallbladder, colon, rectum, bladder, soft tissue or nasopharynx.

In some embodiments, a cancer comprising cells having a mutation which results in increased expression of a ligand for HER3 (e.g. comprising cells having an NRG gene fusion, e.g. an NRG1 gene fusion or an NRG2 gene fusion) is selected from: lung cancer, non-small cell lung cancer, lung adenocarcinoma, invasive mucinous lung adenocarcinoma, lung squamous cell carcinoma, breast cancer, breast carcinoma, breast invasive carcinoma, head and neck cancer, head and neck squamous cell carcinoma, renal cancer, renal clear cell carcinoma, ovarian cancer, ovarian serous cystadenocarcinoma, pancreatic cancer, pancreatic adenocarcinoma, pancreatic ductal adenocarcinoma, prostate cancer, prostate adenocarcinoma, endometrial cancer, uterine carcinosarcoma, gallbladder cancer, cholangiocarcinoma, colorectal cancer, bladder cancer, urothelial bladder cancer, sarcoma, soft tissue sarcoma, neuroendocrine tumor and neuroendocrine tumor of the nasopharynx.

In particular embodiments, the cancer to be treated in accordance with the present invention is lung cancer (e.g. non-small cell lung cancer, lung adenocarcinoma, invasive mucinous lung adenocarcinoma or lung squamous cell carcinoma) comprising cells having an NRG1 gene fusion.

It will be appreciated that in embodiments herein, cancers comprising cells having specified characteristics may be or comprise tumors comprising cells having those characteristics.

As is common in the art, a cancer/tumor comprising cells having specified characteristics may be referred to herein simply as a cancer/tumor having those characteristics. By way of illustration, a cancer/tumor comprising cells having an NRG1 gene fusion may be referred to simply as “a cancer/tumor comprising NRG1 gene fusion”, or “an NRG1 gene fusion cancer/tumor”.

Administration of the antigen-binding molecules and compositions described herein is preferably in a “therapeutically effective” or “prophylactically effective” amount, this being sufficient to show therapeutic or prophylactic benefit to the subject. The actual amount administered, and rate and time-course of administration, will depend on the nature and severity of the disease/condition and the particular article administered. Prescription of treatment, e.g. decisions on dosage etc., is within the responsibility of general practitioners and other medical doctors, and typically takes account of the disease/disorder to be treated, the condition of the individual subject, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins. Administration may be alone or in combination with other treatments, either simultaneously or sequentially dependent upon the condition to be treated. The antigen-binding molecule or composition described herein and a therapeutic agent may be administered simultaneously or sequentially.

In some embodiments, the methods comprise additional therapeutic or prophylactic intervention, e.g. for the treatment/prevention of a cancer. In some embodiments, the therapeutic or prophylactic intervention is selected from chemotherapy, immunotherapy, radiotherapy, surgery, vaccination and/or hormone therapy. In some embodiments, the therapeutic or prophylactic intervention comprises leukapheresis. In some embodiments the therapeutic or prophylactic intervention comprises a stem cell transplant.

In some embodiments the antigen-binding molecule or composition of the present disclosure is administered in combination with an agent capable of inhibiting signalling mediated by an EGFR family member.

Accordingly, the invention provides compositions comprising an article according to the present invention (e.g. an antigen-binding molecule or composition disclosed herein) and another agent capable of inhibiting signalling mediated by an EGFR family member (e.g. EGFR, HER2, HER3 or HER4). Also provided is the use of such compositions in methods of medical treatment and prophylaxis of diseases/conditions described herein.

Also provided are methods for treating/preventing diseases/conditions described herein comprising administering an antigen-binding molecule or composition disclosed herein and another agent capable of inhibiting signalling mediated by an EGFR family member.

Agents capable of inhibiting signalling mediated by EGFR family members are known in the art, and include e.g. small molecule inhibitors (e.g. tyrosine kinase inhibitors), monoclonal antibodies (and antigen-binding fragments thereof), peptide/polypeptide inhibitors (e.g. decoy ligands/receptors or peptide aptamers) and nucleic acids (e.g. antisense nucleic acid, splice-switching nucleic acids or nucleic acid aptamers). Inhibitors of signalling mediated by EGFR family members include agents that inhibit signalling through a direct effect on an EGFR family member, an interaction partner therefore, and/or a downstream factor involved in signalling mediated by the EGFR family member.

In some embodiments the antagonist of signalling mediated by an EGFR family member inhibits signalling mediated by one or more of EGFR, HER2, HER4 and HER3. Inhibitors of signalling mediated by EGFR family members are described e.g. in Yamaoka et al., Int. J. Mol. Sci. (2018), 19, 3491, which is hereby incorporated by reference in its entirety. In some embodiments the antagonist is a pan-ErbB inhibitor. In some embodiments the antagonist is an inhibitor of signalling mediated by EGFR (e.g. cetuximab, panitumumab, gefitinib, erlotinib, lapatinib, afatinib, brigatinib, icotinib, osimertinib, zalutumumab, vandetanib, necitumumab, nimotuzumab, dacomitinib, duligotuzumab or matuzumab). In some embodiments the antagonist is an inhibitor of signalling mediated by HER2 (e.g. trastuzumab, pertuzumab, lapatinib, neratinib, afatinib, dacomitinib, MM-111, MCLA-128 or margetuximab). In some embodiments the antagonist is an inhibitor of signalling mediated by HER3 (e.g. seribantumab, lumretuzumab, elgemtumab, KTN3379, AV-203, GSK2849330, REGN1400, MP-RM-1, EV20, duligotuzumab, MM-111, istiratumab, MCLA-128, patritumab, EZN-3920, RB200 or U3-1402). In some embodiments the antagonist is an inhibitor of signalling mediated by HER4 (e.g. lapatinib, ibrutinib, afatinib, dacomitinib or neratinib).

In some embodiments the antagonist of signalling mediated by an EGFR family member inhibits a downstream effector of signalling by an EGFR family member. Downstream effectors of signalling by an EGFR family members include e.g. PI3K, AKT, KRAS, BRAF, MEK/ERK and mTOR. In some embodiments, the antagonist of signalling mediated by an EGFR family member is an inhibitor of the MAPK/ERK pathway. In some embodiments, the antagonist of signalling mediated by an EGFR family member is an inhibitor of the PI3K/ATK/mTOR pathway. In some embodiments the antagonist is a PI3K inhibitor (e.g. pictilisib, buparlisib, idelalisib, copanlisib or duvelisib). In some embodiments the antagonist is an AKT inhibitor (e.g. MK-2206, AZD5363, ipatasertib, VQD-002, perifosine or miltefosine). In some embodiments the antagonist is a BRAF inhibitor (e.g. vemurafenib, dabrafenib, SB590885, XL281, RAF265, encorafenib, GDC-0879, PLX-4720, sorafenib, or LGX818). In some embodiments the antagonist is a MEK/ERK inhibitor (e.g. trametinib, cobimetinib, binimetinib, selumetinib, PD-325901, CI-1040, PD035901, or TAK-733). In some embodiments the antagonist is a mTOR inhibitor (e.g. rapamycin, deforolimus, temsirolimus, everolimus, ridaforolimus or sapanisertib).

In some embodiments, the cancer to be treated/prevented in accordance with the present disclosure (including monotherapy or combination therapy) is a cancer which is resistant to treatment with an antagonist of signalling mediated by an EGFR family member (e.g. EGFR, HER2, HER4 and/or HER3), e.g. an antagonist as described in the preceding three paragraphs. In some embodiments the subject to be treated has a cancer which is resistant to treatment with an antagonist of signalling mediated by an EGFR family member. In some embodiments the subject to be treated has a cancer which has developed resistance to treatment with an antagonist of signalling mediated by an EGFR family member. In some embodiments the subject to be treated has a cancer which previously responded to treatment with an antagonist of signalling mediated by an EGFR family member, and which is now resistant to treatment with the antagonist. In some embodiments the subject to be treated has a cancer which has relapsed and/or progressed following treatment with an antagonist of signalling mediated by an EGFR family member. In some embodiments the subject to be treated has a cancer which initially responded to treatment with an antagonist of signalling mediated by an EGFR family member, but later progressed on said treatment.

The skilled person is readily able to identify cancers and subjects according to the preceding paragraph. Such cancers and subjects may be identified e.g. through monitoring of the development/progression of the cancer (and/or correlates thereof) over time e.g. during the course of treatment with an antagonist of signalling mediated by an EGFR family member. In some embodiments, identification of such subjects/cancers may comprise analysis of a sample (e.g. a biopsy), e.g. in vitro. In some embodiments the cancer may be determined to comprise cells having a mutation which is associated with reduced susceptibility and/or resistance to treatment with the antagonist. In some embodiments the cancer may be determined to comprise cells having upregulated expression of an EGFR family member.

In particular embodiments, the cancer to be treated is a cancer which is resistant to treatment with an antagonist of signalling mediated by EGFR and/or HER2. In some embodiments the subject to be treated has a cancer which is resistant to treatment with an antagonist of signalling mediated by EGFR and/or HER2. In some embodiments the subject to be treated has a cancer which has developed resistance to treatment with an antagonist of signalling mediated by EGFR and/or HER2. In some embodiments the subject to be treated has a cancer which previously responded to treatment with an antagonist of signalling mediated by EGFR and/or HER2, and which is now resistant to treatment with the antagonist. In some embodiments the subject to be treated has a cancer which has relapsed and/or progressed following treatment with an antagonist of signalling mediated by EGFR and/or HER2. In some embodiments the subject to be treated has a cancer which initially responded to treatment with an antagonist of signalling mediated by EGFR and/or HER2, but later progressed on said treatment.

In particular embodiments, the cancer to be treated comprises mutation conferring resistance to treatment with an inhibitor of BRAF. In some embodiments, the mutation is mutation at BRAF V600. In some embodiments, the mutation is BRAF V600E or V600K.

In particular embodiments, the cancer to be treated comprises mutation conferring resistance to treatment with an inhibitor of BRAF (e.g. mutation at BRAF V600), and the treatment comprises administration of vemurafenib or darafenib.

In some embodiments the antigen-binding molecule or composition described herein is administered in combination with an agent capable of inhibiting signalling mediated by an immune checkpoint molecule. In some embodiments the immune checkpoint molecule is e.g. PD-1, CTLA-4, LAG-3, VISTA, TIM-3, TIGIT or BTLA. In some embodiments the antigen-binding molecule or composition described herein is administered in combination with an agent capable of promoting signalling mediated by a costimulatory receptor. In some embodiments the costimulatory receptor is e.g. CD28, CD80, CD40L, CD86, OX40, 4-1 BB, CD27 or ICOS.

Accordingly, the invention provides compositions comprising an antigen-binding molecule or composition according to the present disclosure and an agent capable of inhibiting signalling mediated by an immune checkpoint molecule. Also provided are compositions comprising an antigen-binding molecule or composition according to the present disclosure and an agent capable of promoting signalling mediated by a costimulatory receptor. Also provided is the use of such compositions in methods of medical treatment and prophylaxis of diseases/conditions described herein.

Also provided are methods for treating/preventing diseases/conditions described herein comprising administering an antigen-binding molecule or composition according to the present disclosure and an agent capable of inhibiting signalling mediated by an immune checkpoint molecule. Also provided are methods for treating/preventing diseases/conditions described herein comprising administering an antigen-binding molecule or composition according to the present disclosure and an agent capable of promoting signalling mediated by a costimulatory receptor.

Agents capable of inhibiting signalling mediated by immune checkpoint molecules are known in the art, and include e.g. antibodies capable of binding to immune checkpoint molecules or their ligands, and inhibiting signalling mediated by the immune checkpoint molecule. Other agents capable of inhibiting signalling mediated by an immune checkpoint molecule include agents capable of reducing gene/protein expression of the immune checkpoint molecule or a ligand for the immune checkpoint molecule (e.g. through inhibiting transcription of the gene(s) encoding the immune checkpoint molecule/ligand, inhibiting post-transcriptional processing of RNA encoding the immune checkpoint molecule/ligand, reducing stability of RNA encoding the immune checkpoint molecule/ligand, promoting degradation of RNA encoding the immune checkpoint molecule/ligand, inhibiting post-translational processing of the immune checkpoint molecule/ligand, reducing stability the immune checkpoint molecule/ligand, or promoting degradation of the immune checkpoint molecule/ligand), and small molecule inhibitors.

Agents capable of promoting signalling mediated by costimulatory receptors are known in the art, and include e.g. agonist antibodies capable of binding to costimulatory receptors and triggering or increasing signalling mediated by the costimulatory receptor. Other agents capable of promoting signalling mediated by costimulatory receptors include agents capable of increasing gene/protein expression of the costimulatory receptor or a ligand for the costimulatory receptor (e.g. through promoting transcription of the gene(s) encoding the costimulatory receptor/ligand, promoting post-transcriptional processing of RNA encoding the costimulatory receptor/ligand, increasing stability of RNA encoding the costimulatory receptor/ligand, inhibiting degradation of RNA encoding the costimulatory receptor/ligand, promoting post-translational processing of the costimulatory receptor/ligand, increasing stability the costimulatory receptor/ligand, or inhibiting degradation of the costimulatory receptor/ligand), and small molecule agonists.

In particular embodiments, an antigen-binding molecule or composition according to the present disclosure is administered in combination with an agent capable of inhibiting signalling mediated by PD-1.

The agent capable of inhibiting signalling mediated by PD-1 may be a PD-1- or PD-L1-targeted agent.

The agent capable of inhibiting signalling mediated by PD-1 may e.g. be an antibody capable of binding to PD-1 or PD-L1 and inhibiting PD-1-mediated signalling.

In some embodiments, an antigen-binding molecule or composition according to the present disclosure is administered in combination with an agent capable of inhibiting signalling mediated by CTLA-4. The agent capable of inhibiting signalling mediated by CTLA-4 may be a CTLA-4-targeted agent, or an agent targeted against a ligand for CTLA-4 such as CD80 or CD86. In some embodiments, the agent capable of inhibiting signalling mediated by CTLA-4 may e.g. be an antibody capable of binding to CTLA-4, CD80 or CD86 and inhibiting CTLA-4-mediated signalling.

In some embodiments, an antigen-binding molecule or composition according to the present disclosure is administered in combination with an agent capable of inhibiting signalling mediated by LAG-3. The agent capable of inhibiting signalling mediated by LAG-3 may be a LAG-3-targeted agent, or an agent targeted against a ligand for LAG-3 such as MHC class II. In some embodiments, the agent capable of inhibiting signalling mediated by LAG-3 may e.g. be an antibody capable of binding to LAG-3 or MHC class II and inhibiting LAG-3-mediated signalling.

In some embodiments, an antigen-binding molecule or composition according to the present disclosure is administered in combination with an agent capable of inhibiting signalling mediated by VISTA. The agent capable of inhibiting signalling mediated by VISTA may be a VISTA-targeted agent, or an agent targeted against a ligand for VISTA such as VSIG-3 or VSIG-8. In some embodiments, the agent capable of inhibiting signalling mediated by VISTA may e.g. be an antibody capable of binding to VISTA, VSIG-3 or VSIG-8 and inhibiting VISTA-mediated signalling.

In some embodiments, an antigen-binding molecule or composition according to the present disclosure is administered in combination with an agent capable of inhibiting signalling mediated by TIM-3. The agent capable of inhibiting signalling mediated by TIM-3 may be a TIM-3-targeted agent, or an agent targeted against a ligand for TIM-3 such as Galectin 9. In some embodiments, the agent capable of inhibiting signalling mediated by TIM-3 may e.g. be an antibody capable of binding to TIM-3 or Galectin 9 and inhibiting TIM-3-mediated signalling.

In some embodiments, an antigen-binding molecule or composition according to the present disclosure is administered in combination with an agent capable of inhibiting signalling mediated by TIGIT. The agent capable of inhibiting signalling mediated by TIGIT may be a TIGIT-targeted agent, or an agent targeted against a ligand for TIGIT such as CD113, CD112 or CD155. In some embodiments, the agent capable of inhibiting signalling mediated by TIGIT may e.g. be an antibody capable of binding to TIGIT, CD113, CD112 or CD155 and inhibiting TIGIT-mediated signalling.

In some embodiments, an antigen-binding molecule or composition according to the present disclosure is administered in combination with an agent capable of inhibiting signalling mediated by BTLA. The agent capable of inhibiting signalling mediated by BTLA may be a BTLA-targeted agent, or an agent targeted against a ligand for BTLA such as HVEM. In some embodiments, the agent capable of inhibiting signalling mediated by BTLA may e.g. be an antibody capable of binding to BTLA or HVEM and inhibiting BTLA-mediated signalling.

In some embodiments methods employing a combination of an antigen-binding molecule or composition of the present disclosure and an agent capable of inhibiting signalling mediated by an immune checkpoint molecule (e.g. PD-1) provide an improved treatment effect as compared to the effect observed when either agent is used as a monotherapy. In some embodiments the combination provides a synergistic (i.e. super-additive) treatment effect.

Simultaneous administration refers to administration of an antigen-binding molecule or composition according to the present disclosure and therapeutic agent together, for example as a pharmaceutical composition containing both agents (combined preparation), or immediately after each other and optionally via the same route of administration, e.g. to the same artery, vein or other blood vessel. Sequential administration refers to administration of one of the antigen-binding molecule/composition or therapeutic agent followed after a given time interval by separate administration of the other agent. It is not required that the two agents are administered by the same route, although this is the case in some embodiments. The time interval may be any time interval.

Chemotherapy and radiotherapy respectively refer to treatment of a cancer with a drug or with ionising radiation (e.g. radiotherapy using X-rays or y-rays). The drug may be a chemical entity, e.g. small molecule pharmaceutical, antibiotic, DNA intercalator, protein inhibitor (e.g. kinase inhibitor), or a biological agent, e.g. antibody, antibody fragment, aptamer, nucleic acid (e.g. DNA, RNA), peptide, polypeptide, or protein. The drug may be formulated as a pharmaceutical composition or medicament.

The formulation may comprise one or more drugs (e.g. one or more active agents) together with one or more pharmaceutically acceptable diluents, excipients or carriers.

A treatment may involve administration of more than one drug. A drug may be administered alone or in combination with other treatments, either simultaneously or sequentially dependent upon the condition to be treated. For example, the chemotherapy may be a co-therapy involving administration of two drugs, one or more of which may be intended to treat the cancer.

The chemotherapy may be administered by one or more routes of administration, e.g. parenteral, intravenous injection, oral, subcutaneous, intradermal or intratumoral.

The chemotherapy may be administered according to a treatment regime. The treatment regime may be a pre-determined timetable, plan, scheme or schedule of chemotherapy administration which may be prepared by a physician or medical practitioner and may be tailored to suit the patient requiring treatment.

The treatment regime may indicate one or more of: the type of chemotherapy to administer to the patient; the dose of each drug or radiation; the time interval between administrations; the length of each treatment; the number and nature of any treatment holidays, if any etc. For a co-therapy a single treatment regime may be provided which indicates how each drug is to be administered.

Chemotherapeutic drugs may be selected from: Abemaciclib, Abiraterone Acetate, Abitrexate (Methotrexate), Abraxane (Paclitaxel Albumin-stabilized Nanoparticle Formulation), ABVD, ABVE, ABVE-PC, AC, Acalabrutinib, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin (Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axicabtagene Ciloleucel, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Calquence (Acalabrutinib), Campath (Alemtuzumab), Camptosar (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), [No Entries], Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Valrubicin, Valstar (Valrubicin), Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yescarta (Axicabtagene Ciloleucel), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib) and Zytiga (Abiraterone Acetate).

In some embodiments the antigen-binding molecule antigen-binding molecule or composition of the present disclosure is administered in combination with one or more of: trastuzumab, cetuximab, cisplatin, 5-FU or capecitabine. In some embodiments the antigen-binding molecule of the invention is administered in combination with trastuzumab and cisplatin, and 5-FU or capecitabine.

In some embodiments the antigen-binding molecule antigen-binding molecule or composition of the present disclosure is administered in combination with cetuximab. Administration in combination with cetuximab is contemplated in particular for the treatment of head and neck cancer (e.g. head and neck squamous cell carcinoma).

Multiple doses of the antigen-binding molecule or composition described herein may be provided. One or more, or each, of the doses may be accompanied by simultaneous or sequential administration of another therapeutic agent.

Multiple doses may be separated by a predetermined time interval, which may be selected to be one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days, or 1, 2, 3, 4, 5, or 6 months. By way of example, doses may be given once every 7, 14, 21 or 28 days (plus or minus 3, 2, or 1 days).

### Methods of Detection

The present disclosure also provides the antigen-binding molecules and compositions described herein for use in methods for detecting, localizing or imaging HER3, or cells expressing HER3. The antigen-binding molecules described herein may be used in methods that involve binding of the antigen-binding molecule to HER3. Such methods may involve detection of the bound complex of the antigen-binding molecule and HER3.

In particular, detection of HER3 may be useful in methods of diagnosing/prognosing a disease/condition in which cells expressing HER3 are pathologically implicated, identifying subjects at risk of developing such diseases/conditions, and/or may be useful in methods of predicting a subject's response to a therapeutic intervention.

As such, a method is provided, comprising contacting a sample containing, or suspected to contain, HER3 with an antigen-binding molecule described herein, and detecting the formation of a complex of the antigen-binding molecule and HER3. Also provided is a method comprising contacting a sample containing, or suspected to contain, a cell expressing HER3 with an antigen-binding molecule described herein and detecting the formation of a complex of the antigen-binding molecule and a cell expressing HER3.

A sample may be taken from any tissue or bodily fluid. The sample may comprise or may be derived from: a quantity of blood; a quantity of serum derived from the individual's blood which may comprise the fluid portion of the blood obtained after removal of the fibrin clot and blood cells; a tissue sample or biopsy; pleural fluid; cerebrospinal fluid (CSF); or cells isolated from said individual. In some embodiments, the sample may be obtained or derived from a tissue or tissues which are affected by the disease/condition (e.g. tissue or tissues in which symptoms of the disease manifest, or which are involved in the pathogenesis of the disease/condition).

Suitable method formats are well known in the art, including immunoassays such as sandwich assays, e.g. ELISA. The methods may involve labelling the antigen-binding molecule, or target(s), or both, with a detectable moiety, e.g. a fluorescent label, phosphorescent label, luminescent label, immuno-detectable label, radiolabel, chemical, nucleic acid or enzymatic label as described herein. Detection techniques are well known to those of skill in the art and can be selected to correspond with the labelling agent.

Methods of this kind may provide the basis of methods for the diagnostic and/or prognostic evaluation of a disease or condition, e.g. a cancer. Such methods may be performed in vitro on a patient sample, or following processing of a patient sample. Once the sample is collected, the patient is not required to be present for the in vitro method to be performed, and therefore the method may be one which is not practised on the human or animal body. In some embodiments the method is performed in vivo.

Detection in a sample may be used for the purpose of diagnosis of a disease/condition (e.g. a cancer), predisposition to a disease/condition, or for providing a prognosis (prognosticating) for a disease/condition, e.g. a disease/condition described herein. The diagnosis or prognosis may relate to an existing (previously diagnosed) disease/condition.

The present disclosure also provides methods for selecting/stratifying a subject for treatment with a HER3-targeted agent. In some embodiments a subject is selected for treatment/prevention in accordance with the present disclosure, or is identified as a subject which would benefit from such treatment/prevention, based on detection/quantification of HER3, or cells expressing HER3, e.g. in a sample obtained from the subject.

Such methods may involve detecting or quantifying HER3 and/or cells expressing HER3, e.g. in a patient sample. Where the method comprises quantifying the relevant factor, the method may further comprise comparing the determined amount against a standard or reference value as part of the diagnostic or prognostic evaluation. Other diagnostic/prognostic tests may be used in conjunction with those described herein to enhance the accuracy of the diagnosis or prognosis or to confirm a result obtained by using the tests described herein.

Where an increased level of HER3 is detected, or where the presence of—or an increased number/proportion of—cells expressing HER3 is detected in a sample obtained from a subject, the subject may be diagnosed as having a disease/condition in which HER3-expressing cells are pathologically implicated, or being at risk of developing such a disease/condition. In such methods, an “increased” level of expression or number/proportion of cells refers to a level/number/proportion which is greater than the level/number/proportion determined for an appropriate control condition, such as the level/number/proportion detected in a comparable sample (e.g. a sample of the same kind, e.g. obtained from the same fluid, tissue, organ etc.), e.g. obtained from a healthy subject.

Where an increased level of HER3 is detected, or where the presence of—or an increased number/proportion of—cells expressing HER3 is detected in a sample obtained from a subject, the subject may be determined to have a poorer prognosis as compared to a subject determined to have a lower level of HER3, or a reduced number/proportion of cells expressing HER3 in a comparable sample (e.g. a sample of the same kind, e.g. obtained from the same fluid, tissue, organ etc.).

The diagnostic and prognostic methods of the present disclosure may be performed on samples obtained from a subject at multiple time points throughout the course of the disease and/or treatment, and may be used to monitor development of the disease/condition over time, e.g. in response to treatment administered to the subject. The results of characterisation in accordance with the methods may be used to inform clinical decisions as to when and what kind of therapy to administer to a subject.

Methods of diagnosis or prognosis may be performed in vitro on a sample obtained from a subject, or following processing of a sample obtained from a subject. Once the sample is collected, the patient is not required to be present for the in vitro method of diagnosis or prognosis to be performed and therefore the method may be one which is not practised on the human or animal body.

### Subjects

The subject in accordance with aspects described herein may be any animal or human. The subject is preferably mammalian, more preferably human. The subject may be a non-human mammal, but is more preferably human. The subject may be male or female. The subject may be a patient. A subject may have been diagnosed with a disease or condition requiring treatment (e.g. a cancer), may be suspected of having such a disease/condition, or may be at risk of developing/contracting such a disease/condition.

In some embodiments, the subject to be treated according to a therapeutic or prophylactic method of the present disclosure herein is a subject having, or at risk of developing, a cancer. In embodiments according to the present disclosure, a subject may be selected for treatment according to the methods based on characterisation for certain markers of such disease/condition.

### Kits

The present disclosure also provides a kit of parts comprising a cell, or a population of cells, of the cell line deposited 7 May 2021 as ATCC Patent Deposit Number PTA-127062, a composition comprising such a cell or population of cells, antigen-binding molecule(s) expressed from a cell, or a population of cells, of the cell line deposited 7 May 2021 as ATCC Patent Deposit Number PTA-127062, or a pharmaceutical composition comprising such antigen-binding molecule(s). In some embodiments the kit may have at least one container having a predetermined quantity of the relevant article.

In some embodiments, the kit of parts may comprise materials for producing an antigen-binding molecule or composition according to the present disclosure by expression from a cell, or a population of cells, of the cell line deposited 7 May 2021 as ATCC Patent Deposit Number PTA-127062.

In some embodiments, the kit of parts comprises an antigen-binding molecule or composition according to the present disclosure together with instructions for administration to a patient in order to treat a specified disease/condition (e.g. a disease/condition described herein).

In some embodiments the kit of parts may further comprise at least one container having a predetermined quantity of another therapeutic agent (e.g. anti-infective agent or chemotherapy agent). In such embodiments, the kit may also comprise a second medicament or pharmaceutical composition such that the two medicaments or pharmaceutical compositions may be administered simultaneously or separately such that they provide a combined treatment for the specific disease or condition.

### Sequences

| SEQ | | |
|---|---|---|
| ID | | |
| NO: | DESCRIPTION | SEQUENCE |
| 1 | 10D1F hIgG1 HC | QVQLQESGPGLVKPSQTLSLTCTVSGYSITSGYSWHWIRQHPGKGLEWIGSIRYSGGTDYNPSLK |
| | | SLVTISADTSKNQFSLKLSSVTAADTAVYYCARMTTAPWYPFDYWGQGTTVTVSSASTKGPSVFP |
| | | LAPSSKSTSGGTAALGCLVKDYFPEPVTVSWNSGALTSGVHTFPAVLQSSGLYSLSSVVTVPSSS |
| | | LGTQTYICNVNHKPSNTKVDKKVEPKSCDKTHTCPPCPAPELLGGPSVFLFPPKPKDTLMISRTPE |
| | | VTCVVVDVSHEDPEVKFNWYVDGVEVHNAKTKPREEQYNSTYRVVSVLTVLHQDWLNGKEYKC |
| | | KVSNKALPAPIEKTISKAKGQPREPQVYTLPPSRDELTKNQVSLTCLVKGFYPSDIAVEWESNGQP |
| | | ENNYKTTPPVLDSDGSFFLYSKLTVDKSRWQQGNVFSCSVMHEALHNHYTQKSLSLSPGK |
| 2 | 10D1F K LC | DIQMTQSPSSVSASVGDRVTITCKASQIVGSNVAWYQQKPGKAPEPLIYSASYLYSGVPSRFSGS |
| | | GSGTDFTLTISSLQPEDFATYYCQQYSSHPLTFGQGTKLEIKRTVAAPSVFIFPPSDEQLKSGTASV |
| | | VCLLNNFYPREAKVQWKVDNALQSGNSQESVTEQDSKDSTYSLSSTLTLSKADYEKHKVYACEV |
| | | THQGLSSPVTKSFNRGEC |
| 3 | 10D1F hIgG1 HC | CAGGTGCAGCTGCAGGAGTCCGGACCAGGACTGGTGAAGCCATCCCAGACCCTGTCTCTGA |
| | nucleotide | CCTGCACAGTGTCCGGCTACTCCATCACATCTGGCTATAGCTGGCACTGGATCAGGCAGCAC |
| | | CCTGGCAAGGGCCTGGAGTGGATCGGCAGCATCCGCTACTCCGGCGGCACCGACTATAATC |
| | | CCTCTCTGAAGAGCCTGGTGACCATCTCCGCGGACACATCTAAGAACCAGTTCTCCCTGAAG |
| | | CTGAGCTCCGTGACCGCCGCTGATACAGCCGTGTACTATTGTGCTCGGATGACCACAGCCCC |
| | | CTGGTACCCTTTTGACTATTGGGGCCAGGGCACCACAGTGACAGTGTCTAGCGCTAGCACCA |
| | | AGGGCCCATCGGTCTTCCCCCTGGCACCCTCCTCCAAGAGCACCTCTGGGGGCACAGCGGC |
| | | CCTGGGCTGCCTGGTCAAGGACTACTTCCCCGAACCGGTGACGGTGTCATGGAACTCAGGC |
| | | GCCCTGACCAGCGGCGTGCACACCTTCCCGGCTGTCCTACAGTCCTCAGGACTCTACTCCCT |
| | | CAGCAGCGTGGTGACCGTGCCCTCCAGCAGCTTGGGCACCCAGACCTACATCTGCAACGTG |
| | | AATCACAAGCCCAGCAACACCAAGGTGGACAAGAAAGTTGAGCCCAAATCTTGTGACAAAAC |
| | | TCACACATGCCCACCGTGCCCAGCACCTGAACTCCTGGGGGGACCGTCAGTCTTCCTCTTCC |
| | | CCCCAAAACCCAAGGACACCCTCATGATCTCCCGGACCCCTGAGGTCACATGCGTGGTGGT |
| | | GGACGTGAGCCACGAAGACCCTGAGGTCAAGTTCAACTGGTACGTGGACGGCGTGGAGGTG |
| | | CATAATGCCAAGACAAAGCCCCGGGAGGAGCAGTACAACAGCACGTACCGTGTGGTCAGCG |
| | | TCCTCACCGTCCTGCACCAGGACTGGCTGAATGGCAAGGAGTACAAGTGCAAGGTCTCCAAC |
| | | AAAGCCCTCCCAGCCCCCATCGAGAAAACCATCTCCAAAGCCAAAGGGCAGCCCCGAGAAC |
| | | CACAGGTGTACACCCTGCCCCCATCCCGGGATGAGCTGACCAAGAACCAGGTCAGCCTGAC |
| | | CTGCCTGGTCAAAGGCTTCTATCCCAGCGACATCGCCGTGGAGTGGGAGAGCAATGGGCAG |
| | | CCGGAGAACAACTACAAGACCACGCCTCCCGTGCTGGACTCCGACGGCTCCTTCTTCCTCTA |
| | | CAGCAAGCTCACCGTGGACAAGAGCAGGTGGCAGCAGGGGAACGTCTTCTCATGCTCCGTG |
| | | ATGCATGAGGCTCTGCACAACCACTACACGCAGAAGAGCCTCTCCCTGTCTCCGGGTAAA |
| 4 | 10D1F K LC | GACATCCAGATGACCCAGTCTCCTAGCTCCGTGAGCGCCTCCGTGGGCGATAGGGTGACCA |
| | nucleotide | TCACATGCAAGGCCAGCCAGATCGTGGGCTCCAACGTGGCTTGGTACCAGCAGAAGCCCGG |
| | | CAAGGCCCCTGAGCCGCTGATCTATTCCGCTTCATACCTGTATAGCGGAGTGCCATCCAGGT |
| | | TCAGCGGCTCCGGCTCTGGAACCGACTTTACCCTGACAATCAGCTCCCTGCAGCCAGAGGAT |
| | | TTCGCTACATATTATTGTCAGCAGTACTCTAGCCACCCCCTGACCTTTGGCCAGGGCACAAAG |
| | | CTGGAGATCAAGCGTACGGTGGCGGCGCCATCTGTCTTCATCTTCCCGCCATCTGATGAGCA |
| | | GTTGAAATCTGGAACTGCCTCTGTTGTGTGCCTGCTGAATAACTTCTATCCCAGAGAGGCCAA |
| | | AGTACAGTGGAAGGTGGATAACGCCCTCCAATCGGGTAACTCCCAGGAGAGTGTCACAGAG |
| | | CAGGACAGCAAGGACAGCACCTACAGCCTCAGCAGCACCCTGACGCTGAGCAAAGCAGACT |
| | | ACGAGAAACACAAAGTCTACGCCTGCGAAGTCACCCATCAGGGCCTGAGCTCGCCCGTCACA |
| | | AAGAGCTTCAACAGGGGAGAGTGT |

The present disclosure includes the combination of the aspects and preferred features described except where such a combination is clearly impermissible or expressly avoided.

The section headings used herein are for organizational purposes only and are not to be construed as limiting the subject matter described.

Aspects and embodiments of the present disclosure will now be illustrated, by way of example, with reference to the accompanying figures. Further aspects and embodiments will be apparent to those skilled in the art. All documents mentioned in this text are incorporated herein by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

Where a nucleic acid sequence is disclosed or referred to herein, the reverse complement thereof is also expressly contemplated.

Methods described herein may preferably be performed in vitro. The term “in vitro” is intended to encompass procedures performed with cells in culture whereas the term “in vivo” is intended to encompass procedures with/on intact multi-cellular organisms.

### BRIEF DESCRIPTION OF THE FIGURES

Embodiments and experiments illustrating the principles of the present disclosure will now be discussed with reference to the accompanying figures.

### EXAMPLES

In the following Examples, the inventors describe the generation and characterisation of a novel cell line expressing a HER3-binding antibody.

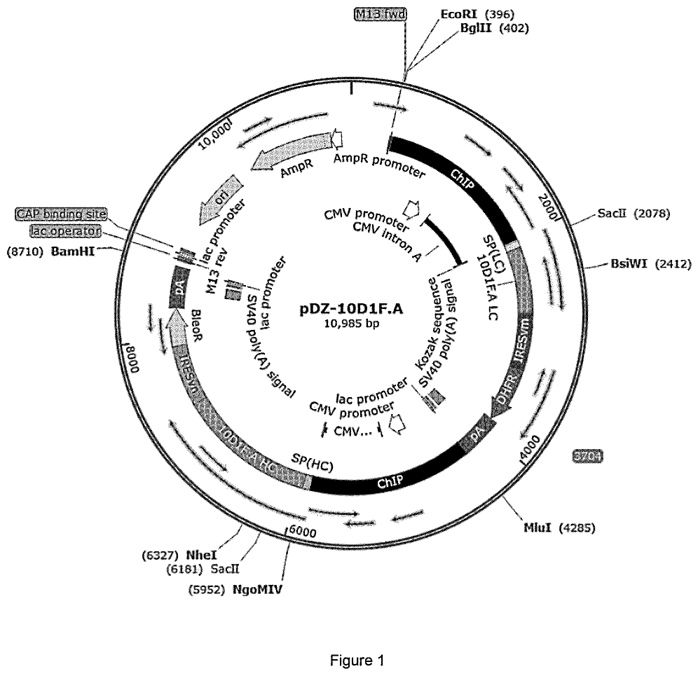

FIG. 1. Schematic representation of the expression vector pDZ-10D1 F.A encoding 10D1 F hIgG1. Sequence features of the vector are described at Example 2.2.

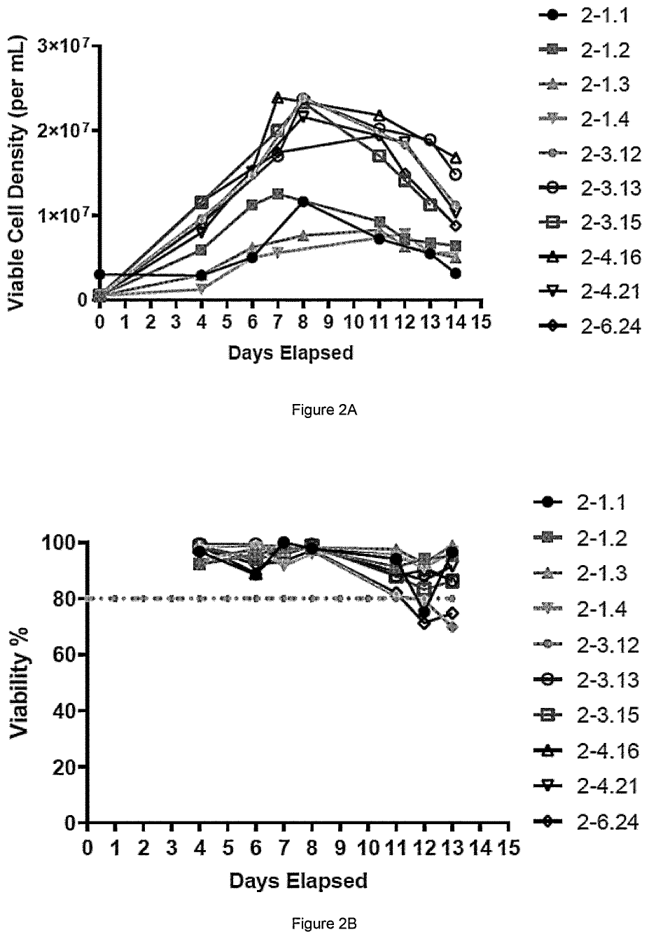

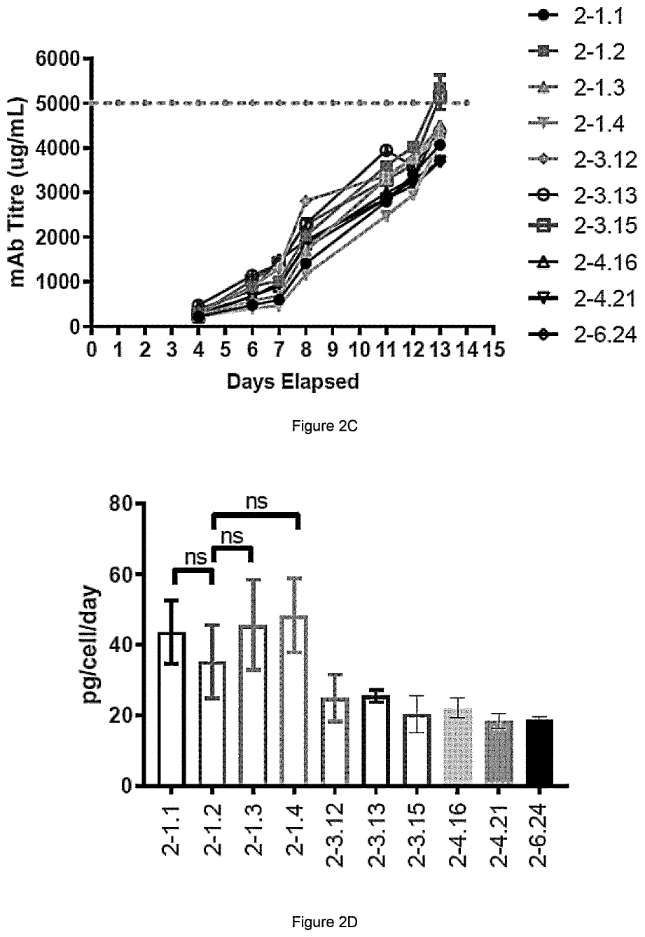

FIGS. 2A to 2D. Graphs and bar chart showing (2A) the viable cell density per ml, (2B) the percentage of viable cells in culture, (2C) the monoclonal antibody titer in the cell culture supernatant in μg/ml, and (2D) cell specific productivity expressed in pg/cell/day (qP), for clones 2-1.1, 2-1.2, 2-1.3, 2-1.4, 2-3.12, 2-3.13, 2-3.15, 2-4.16, 2-4.21 and 2-6.24, in a 14-day fed batch process in 45 ml of medium comprising EX-CELL Advanced CHO Fed-Batch Medium+6 mM L-Glutamine+250 nM MTX.

FIGS. 3A and 3B. Graphs showing (3A) the viable cell density per ml and monoclonal antibody titer in the cell culture supernatant in μg/ml for clone 2-3.12 on the indicated days, and (3B) doubling time of the cells in culture, over the course of a 15-day culture in a 3 L SmartGlass bioreactor, in cell culture medium comprising EX-CELL Advanced CHO Fed-Batch Medium+6 mM L-Glutamine+250 nM MTX.

### Example 1: 10D1F

### 1.1 Characterisation of 10D1F in WO 2019/185878 A1 and WO 2021/048274 A1

The HER3-binding antibody clone designated 10D1F is described in WO 2019/185878 A1 (incorporated by reference in its entirety).

10D1 F comprises the heavy chain variable region shown in SEQ ID NO:36 of WO 2019/185878 A1, and the light chain variable region shown in SEQ ID NO:83 of WO 2019/185878 A1 (also referred to therein as ‘10D1_c89’). Example 2.2 of WO 2019/185878 A1 describes a molecule (molecule [16]) comprising the VH and VL regions of 10D1 F in human IgG1/Vκ format, formed of SEQ ID NO:206 of WO 2019/185878 A1 and SEQ ID NO:207 of WO 2019/185878 A1 (10D1F hIgG1).

Examples 8.1 to 8.3 and FIGS. 42 to 46 of WO 2019/185878 A1 show that 10D1 F hIgG1 binds to human HER3 with high affinity and specificity (displaying no cross-reactivity with other human EGFR family members), while retaining high-affinity binding to cyano, mouse and rat HER3.

Example 8.6 and FIGS. 49A and 49B of WO 2019/185878 A1 demonstrate that 10D1 F hIgG1 binds to HER3 in a ligand (NRG)-independent fashion, and through a topologically distant epitope of HER3 to the epitope bound by anti-HER3 antibodies M-05-74 and M-08-11. Example 8.10 and FIG. 78 of WO 2021/048274 A1 demonstrate that 10D1F hIgG1 binds to human HER3 with subpicomolar affinity in the presence or absence of human NRG1.

Example 4.1 and FIG. 65, and Example 8.7 and FIG. 52 of WO 2019/185878 A1 demonstrate that 10D1F hIgG1 is highly potent at inhibiting interaction between HER3 and HER2, and does so in a dose-dependent manner. Example 8.7 and FIG. 53 of WO 2019/185878 A1 show that 10D1 F hIgG1 inhibits interaction between HER3 and EGFR in a dose-dependent fashion.

Example 8.8 and FIG. 54 of WO 2019/185878 A1 show that 10D1F hIgG1 induces ADCC activity against HER3 overexpressing cells in a dose-dependent manner.

Example 8.9 and FIGS. 55, 63 and 64 of WO 2019/185878 A1 demonstrate that 10D1F hIgG1 inhibits HER3-mediated signalling in cells of HER3-expressing cancer cell lines in vitro.

Example 11 and FIG. 71 of WO 2019/185878 A1 show that 10D1 F hIgG1 also inhibits HER3-mediated signalling in HER3-expressing human cancer cell line-derived xenograft tumors in vivo. Example 14 and FIG. 79 of WO 2021/048274 A1 demonstrate that 10D1F is extremely potent at inhibiting growth of xenograft tumors derived from a human cancer cell line harbouring an NRG gene fusion.

Examples 9.3, 9.4 and FIGS. 59, 60, 61, 62, 74 and 77 of WO 2019/185878 A1 demonstrate that 10D1 F potently inhibits the growth of cancer cells in vitro, and also potently inhibits growth of human cancer cell line-derived xenograft tumors in vivo. Example 10 and FIGS. 67 and 68 of WO 2019/185878 A1 show that 10D1F hIgG1 inhibits in vitro proliferation of thyroid cancer cell lines harbouring the V600E BRAF mutation.

Example 12 and FIGS. 72 and 73 of WO 2019/185878 A1 show that 10D1F hIgG1 is not substantially internalised by HER3-expressing cells.

Example 13 and FIGS. 75 and 76 of WO 2019/185878 A1 demonstrate the utility of 10D1F hIgG1 to be employed for the detection of HER3.

Example 8.4 and FIG. 47A of WO 2019/185878 A1 show that 10D1 F hIgG1 is thermostable, having a melting temperature of 70.0° C. as determined by Differential Scanning Fluorimetry.

Example 9.1, 9.2 and FIGS. 56, 57, 58 and 69, 70 of WO 2019/185878 A1 evidence that 10D1 F hIgG1 has favourable pharmacological and toxicological profiles.

### Example 2: Cell Line Development

The present example describes the production of a cell lines stably expressing antibody 10D1 F hIgG1.

### 2.1 Adaptation to Culture in Serum-Free Medium

CHO-k1 cells (ATCC, Cat. No. CCL-61) were first adapted to suspension culture in serum-free medium. Briefly, CHO-k1 cells were first cultured in F-12K medium supplemented with 10% heat-inactivated FBS (F-12K+10 medium).

After two passages, the medium was exchanged to F75-25 medium (comprising 75% F-12K+10 medium, and 25% “50:50 medium”; “50:50 medium” is medium comprising 50% PF CHO Serum-Free Medium+50% CD CHO Serum-Free Medium+6 mM L-Glutamine+0.05% Pluronic F-68) with a seeding density of 5×10⁵ cell/ml. Cells were transferred to shake flasks and cultured in a 37° C., 5% CO₂, humidified incubator with agitation at 110 rpm.

After three passages in F75-25 medium, the cell culture was diluted into F50-50 medium (comprising 50% F-12K+10 medium, and 50% 50:50 medium) at seeding density 5×10⁵ cell/ml. The cells were passaged 6 times, and subsequently diluted into F25-F75 medium (comprising 25% F-12K+10 medium, 75% 50:50 medium) at seeding density 5×10⁵ cell/ml.

After two passages, the cells were diluted into 50:50 medium at seeding density of 5×10⁵ cell/ml and cultured for one passage, before two passages at a seeding density of 2×10⁵ cell/ml in 50:50 medium. At the end of the process of adaption to culture in serum-free medium, the viability of cells in culture was 96.8%.

Cells were then cultured in EX-CELL Advanced CHO Fed-Batch medium (SAFC; Sigma Cat. No. 14366C) supplemented with 6 mM L-Glutamine (Sigma Cat. No. G8540), which is hereafter referred to as ‘EX-CELL medium’. Cells were diluted in EX-CELL medium at seeding density 2×10⁵ cell/ml, and cultured at 37° C. in an 8% CO₂ atmosphere, humidified incubator with agitation at 125 rpm.

### 2.2 Expression Vector Construction

A polycistronic expression vector encoding SEQ ID NO:1 and SEQ ID NO:2 of 10D1F hIgG1 was produced by cloning VH and VL region sequences codon-optimised for expression by CHO cells into MabDZ vector (described e.g. in US 2012/0301919 A1).

The polycistronic vector pDZ-1001 F.A encoding 1001 F hIgG1 is represented schematically in FIG. 1, and its key sequence features are summarised in the table below.

| Element | Description |
|---|---|
| ChIP | Chimeric promoter consisting of murine cytomegalo- |
| | virus (CMV) enhancer (−707 to −286 relative to the |
| | transcriptional start site of +1, with 1 mutation at T- |
| | 599C) (GenBank: M11788.1) |
| | Spel Restriction Site |
| | Human CMV core promoter consisting of human CMV |
| | 1st exon and human CMV intron A (−223 to +953 |
| | relative to the transcriptional start site of +1, with the |
| | following mutations: T-74C, C-70T, G-62A, del211T, |
| | C738G, del740C, C720T, T719G) (GenBank: |
| | M60321.1) |
| IRESvm | Mutated encephalomyocarditis virus (EMCV) internal |
| IRESvn | ribosome entry site (IRES) (GenBank: M81861.1) |
| pA | Simian virus 40 (SV40) polyadenylation signal, |
| | sequence from pcDNA3.1/Zeo+ vector (Thermo |
| | Fisher Scientific) |
| 10D1F.A | Antibody light chain cDNA consisting of light chain |
| LC | variable region (VL) and constant sequence from |
| | human IgG kappa chain (CK) (SEQ ID NO: 4) |
| 10D1F.A | Antibody heavy chain cDNA consisting of heavy chain |
| HC | variable region (VH) and constant sequence from |
| | human IgG1 (SEQ ID NO: 3) |
| DHFR | Dihydrofolate reductase cDNA (GenBank: NM_ |
| | 010049.3) |
| BleoR | Zeocin resistance gene from pUC57 (GenBank: |
| | Y14837.1) |
| Ori. | E. coli origin of replication from pUC57 (GenBank: |
| | Y14837.1) |
| AmpR | Ampicilin resistant gene from pUC57 (GenBank: |
| | Y14837.1) |
| AmpR | Promoter of Ampicilin resistance gene from pUC57 |
| promoter | (GenBank: Y14837.1) |
| Lac promoter | Lac promoter from pUC57 (GenBank: Y14837.1) |
| Lac operator | Lac operator from pUC57 (GenBank: Y14837.1) |

### 2.3 Transfection

Cells were transfected with the vector described in Example 2.2 above.

EX-CELL medium-adapted CHO-k1 cells were thawed and maintained at 37° C., 8% CO₂ humidified incubator, and 125 rpm agitation conditions for one week prior to transfection. 1×10⁷ cells were then seeded at a density of 5×10⁶ cell/ml, and electroporated with 5 μg of linearized expression vector using the 4D-Nucleofector kit (Lonza, Switzerland), electroporation program CA201.

Electroporated cells were incubated at 37° C., 5% CO₂ humidified static cell incubator in 6-well plates containing 2 ml EX-CELL medium for 24 hr. Cells where then harvested by centrifugation and resuspended in selection medium comprising EX-CELL Advanced CHO Fed-Batch Medium+6 mM L-Glutamine+250 nM methotrexate (MTX; Sigma Cat. No. M8407)+200 μg/ml Zeocin, at a seeding density of 5×10⁵ cells/ml.

Cells were transferred to fresh selection medium once per week. After four weeks, cells were transferred to maintenance medium (comprising EX-CELL Advanced CHO Fed-Batch Medium+6 mM L-Glutamine+250 nM MTX).

### 2.4 Generation of Stable Clone

The stable clone was generated by three rounds of limiting dilution from the transfected stable pool produced as described in Example 2.3, in medium comprising: 80% EX-CELL CHO Cloning Medium+6 mM L-Glutamine.

Clonality analysis was calculated theoretically using a Poisson distribution. As a result, the probability of getting monoclonal cells after 3 rounds limiting dilution, with each round seeding density at 0.5 cell/well, is 98.8%.

At the final round of limiting dilution, a total of 10 clones were selected for further characterisation: 2-1.1, 2-1.2, 2-1.3, 2-1.4, 2-3.12, 2-3.13, 2-3.15, 2-4.16, 2-4.21 and 2-6.24.

### Example 3: Cell Line Characterisation

### 3.1 Growth and Productivity

The cell lines were characterized for growth and productivity by a 14-day fed batch process in 45 ml of medium comprising EX-CELL Advanced CHO Fed-Batch Medium+6 mM L-Glutamine+250 nM MTX.

Viable cell densities, percentage viability, monoclonal antibody titer, and cell-specific productivity were determined as follows.

### Analysis of Viability

Viable cell densities (VCD) and percentage viability were determined by analysis using a hemocytometer under a 20× objective lens on an inverted light microscopy, and using the trypan blue exclusion method.

### Analysis of Monoclonal Antibody Titer

Monoclonal antibody titers were determined by quantification using a BLI system (Octet-Protein A), as follows:
Sample preparation
   Cell culture supernatant from Day 4 was diluted 10 times with sterile PBS; giving a total dilution factor of 10
   Cell culture supernatant from Day 7-9 was first diluted 10 times with sterile PBS, followed by a 2 times dilution with 100% assay buffer (10% medium comprising EX-CELL Advanced CHO Fed-Batch Medium with 6 mM L-Gln and 250 nM MTX, with 90% PBS, 0.22 μm filtered); giving a total dilution factor of 20
   Cell culture supernatant from Day 12 was first diluted 10 times with sterile PBS, followed by a 3 times dilution with 100% assay buffer; giving a total dilution factor of 30
   Cell culture supernatant from Day 13 onwards was first diluted 10 times with sterile PBS, followed by a 5 times dilution 100% assay buffer; giving a total dilution factor of 50
Preparation of protein standards
   Purified 10D1F hIgG1 was diluted in assay buffer to the following concentrations: 1, 3, 10, 30, 100, 300, 500 and 700 μg/ml.
Quantification
   Protein A biosensors were pre-hydrated in assay buffer for 10 min.
   Samples and protein standards were transferred to polypropylene 96-well black flat-bottom plates (Greiner Bio-One) at a volume of 200 μl per well, in duplicate. The samples and standard proteins were then transferred to the Octet QK384 system for titer screening using basic quantification with in-plate standards mode.
   Biosensors were first dipped in sample/standard wells for 120 s to obtain the binding rate of antibody to protein A biosensor. Biosensors were regenerated with regeneration buffer (10 mM glycine pH 1.7) for 5 s and washed/equilibrated in assay buffer for another 5 s, this regeneration-equilibrium cycle was repeated 4 times before taking measurements for the next sample.
   Two measurements were taken for each sample, and all measurements were performed at 25° C. under 400 rpm agitation.

### Analysis of Cell-Specific Productivity (qP)

Integrated viable cell density (IVCD, cell/ml) between 2 sampling days was calculated using the formula (VCDb+VCDaa)/2×(b−a), where ‘a’ represents cultivation time (in days) at day a, ‘b’ represents cultivation time (in days) at day b, and b>a. ‘VCDb’ is the cell count at day b, and ‘VCDaa’ is the cell count at day a with a dilution factor of 0.9 reflecting the culture replacement by feed medium. IVCD for day 13 is the sum of each interval IVCD.

Integrated titer at day x is calculated by current titer (μg/ml) in addition with all the lost titer during day 4, 6, 8, 11 that occurred before day x. Integrated titer is further corrected for evaporation effects, the rate of which is estimated at 1.333 ml per day due to an observation of 20% (12 ml) lost on the day 14 of Fed-Batch culture, and assuming evaporation rate is constant during the whole Fed-Batch process.

The cell-specific productivity is plotted with mAb titer against IVCD. The qP (pg/cell/day) of day n is calculated using the formula: (integrated titer on day n)/(IVCD on day n)×1×10⁶.

The results are shown in FIGS. 2A to 2D.

### 3.2 Stability

Clones 2-1.1, 2-1.2, 2-1.3, 2-1.4, 2-3.12, 2-3.13 and 2-3.15 were analysed in order to evaluate their phenotypic stability.

Cell growth and productivity was compared across different generations of the different clones obtained by passaging cells twice per week with a seeding density of 3×10⁵ cell/ml.

The various generations of the different clones were analysed in a 13-day fed batch process in 60 ml of medium comprising EX-CELL Advanced CHO Fed-Batch Medium+6 mM L-Glutamine+250 nM MTX, maintained at 37° C., 80% relative humidity, 8% CO₂ and 125 rpm agitation.

The percentage of viable cells, IVCD, monoclonal antibody titer and qP were determined on the indicated days as described in section 3.1 above.

Antibody product from the 13-day fed batch process was purified using one-step purification with Protein A column, and yield, monomeric purity, and affinity of binding to human HER3 were evaluated.

### Protein A Purification

Clarified antibody-containing cell culture supernatant was loaded onto HiTrap MabSelect SuRe column 5 ml (GE Healthcare, USA) on an AKTA Start chromatography system (GE Healthcare, USA) with loading speed at 1.5 ml/min, followed by 10 column volume wash of 20 mM sodium phosphate buffer with 250 mM NaCl at pH 7.4 and 10 column volume wash of 20 mM sodium phosphate buffer at pH 7.4 and eluted with 0.1 M sodium citrate pH 3.5.

Eluted antibody concentrations were determined by quantification using a Nanodrop A280 and general IgG's extinction coefficient at 1.37. Samples were stored at 4° C. until use.

### Analysis of Aggregation

Purified antibody samples were diluted to a concentration of 0.4 mg/ml in a total volume of 1 ml in a microtube with PBS. 200 μg of the antibody was injected onto a Superdex 200 10/300 GL column (pre-equilibrated with PBS, pH 7.2-7.6). Aggregated and monomeric IgGs were separated under a mobile phase of PBS, which was pumped into column at a constant flow rate of 0.4 min/ml at room temperature, and A280 of the flow through was recorded. Aggregation % was determined using the following formula: (AUC of aggregation peak/AUC of total protein peaks)×100.

### Analysis of Binding to HER3

Anti-human immunoglobulin G (IgG) Fc (AHC)-coated biosensors (Cat #18-5060, Pall ForteBio, Menlo Park, CA) were pre-hydrated with PBST (PBS with 0.05% Tween 20) for 10 min. Samples and assay buffers were dispensed into polypropylene 96-well black flat-bottom plates (Greiner Bio-One, Frickenhausen, Germany) at a volume of 200 μl per well, and then transferred to Octet QK384 system for kinetic screening of mAb-antigen binding.

Biosensors were first dipped in wells containing assay buffer for 60 s to obtain baseline readings, and then IgGs (at a concentration of 12.5 nM) were captured for 120 s, followed by PBST wash for 60 s to remove any unbound antibody or non-specifically-bound protein and the second baseline was collected after IgG capture. Kinetic measurements for antigen binding were performed by dipping each of the antibody-coated biosensors into wells containing a single serial diluted concentration of recombinant human HER3 for 120 s, followed by a 120 s dissociation time by transferring the biosensors into assay buffer containing wells. AHC biosensors were regenerated for 5 s×4 times after each kinetic cycle using 10 mM glycine pH 1.7 to remove bound protein and washed with assay buffer before a new kinetic cycle. Six 1:2 serial dilutions of recombinant human HER3 (at concentrations ranging from 500 nM to 16 nM), were used to ensure an ideal concentration range of antigen, and binding signal spanning the dynamic range of the assay. Measurements for all sensors were generated in real time in parallel. Data analysis was preformed using Octet QK384 analysis 9.0 software (Pall ForteBio) according to manufacturer's recommendations for analysing a high-affinity antigen-antibody kinetics. Responses from all steps were subtracted with reference wells, aligned to baseline, inter-step was corrected at dissociation followed by processing with Savitzky-Golay filtering. The resultant association and dissociation curves were fitted with 1:1 global fitting to obtain values for association rate (kon), dissociation rate (koff), and the equilibrium dissociation constant (Kd). Kd generated from global fitting with R² value above 0.95 is considered reliable.

The results of the analyses are summarised in the tables below.

| | Generation | Generation | Generation |
|---|---|---|---|
| Clone 2-1.1 | 12 | 37 | 54 |
| Averaged peak IVCD (cell/ml) | 7.05E+06 | 9.68E+06 | 1.15E+07 |
| Averaged titer at day 13 (g/l) | 3.006 | 2.959 | 2.762 |
| Averaged IVCD (cell/ml) | 7.83E+07 | 7.82E+07 | 7.92E+07 |
| Averaged qP (pg/cell/day) | 35.05 | 33.35 | 32.16 |
| Averaged viability at day 13 (%) | 97.1 | 87.25 | 82.5 |
| Averaged aggregation (%) | 2.29% | 2.02% | 3.08% |
| Averaged Kd to human HER3 | <1 pM | <1 pM | <1 pM |

| | Generation | Generation | Generation |
|---|---|---|---|
| Clone 2-1.2 | 15 | 57 | 78 |
| Averaged peak IVCD (cell/ml) | 1.36E+07 | 1.23E+07 | 1.19E+07 |
| Averaged titer at day 13 (g/l) | 3.729 | 3.043 | 3.355 |
| Averaged IVCD (cell/ml) | 9.38E+07 | 8.12E+07 | 8.15E+07 |
| Averaged qP (pg/cell/day) | 33.69 | 36.73 | 39.08 |
| Averaged viability at day 13 (%) | 84.65 | 59.45 | 64.1 |
| Averaged aggregation (%) | 4.68% | 3.08% | 2.98% |
| Averaged Kd to human HER3 | <1 pM | <1 pM | <1 pM |

| | Generation | Generation | Generation |
|---|---|---|---|
| Clone 2-1.3 | 10 | 65 | 95 |
| Averaged peak IVCD (cell/ml) | 9.75E+06 | 1.10E+07 | 1.17E+07 |
| Averaged titer at day 13 (g/l) | 3.737 | 3.567 | 3.804 |
| Averaged IVCD (cell/ml) | 7.65E+07 | 8.14E+07 | 9.04E+07 |
| Averaged qP (pg/cell/day) | 42.83 | 41.2 | 36.16 |
| Averaged viability at day 13 (%) | 95.75 | 76.6 | 77.55 |
| Averaged aggregation (%) | 4.87% | 4.50% | 3.50% |
| Averaged Kd to human HER3 | <1 pM | <1 pM | <1 pM |

| | Generation | Generation | Generation |
|---|---|---|---|
| Clone 2-1.4 | 16 | 51 | 74 |
| Averaged peak IVCD (cell/ml) | 1.08E+07 | 1.02E+07 | 1.26E+07 |
| Averaged titer at day 13 (g/l) | 4.322 | 3.909 | 3.56 |
| Averaged IVCD (cell/ml) | 7.50E+07 | 7.93E+07 | 8.56E+07 |
| Averaged qP (pg/cell/day) | 51.25 | 43.21 | 37.35 |
| Averaged viability at day 13 (%) | 85 | 76.9 | 78.7 |
| Averaged aggregation (%) | 5.06% | 3.95% | 3.56% |
| Averaged Kd to human HER3 | <1 pM | <1 pM | <1 pM |

| | Generation | Generation | Generation |
|---|---|---|---|
| Clone 2-3.12 | 19 | 54 | 88 |
| Averaged peak IVCD (cell/ml) | 2.06E+07 | 2.26E+07 | 2.25E+07 |
| Averaged titer at day 13 (g/l) | 4.659 | 4.483 | 4.319 |
| Averaged IVCD (cell/ml) | 1.47E+08 | 1.63E+08 | 1.57E+08 |
| Averaged qP (pg/cell/day) | 28.67 | 25.35 | 24.64 |
| Averaged viability at day 13 (%) | 70.35 | 75.55 | 74.85 |
| Averaged aggregation (%) | 0.98% | 1.53% | 1.38% |
| Averaged Kd to human HER3 | <1 pM | <1 pM | <1 pM |

| | Generation | Generation | Generation |
|---|---|---|---|
| Clone 2-3.13 | 16 | 47 | 77 |
| Averaged peak IVCD (cell/ml) | 1.89E+07 | 1.93E+07 | 2.56E+07 |
| Averaged titer at day 13 (g/l) | 4.256 | 4.023 | 3.918 |
| Averaged IVCD (cell/ml) | 1.33E+08 | 1.33E+08 | 1.49E+08 |
| Averaged qP (pg/cell/day) | 31.98 | 30.07 | 25.34 |
| Averaged viability at day 13 (%) | 75.75 | 76.65 | 71.25 |
| Averaged aggregation (%) | 0.19% | 0.91% | 0.84% |
| Averaged Kd to human HER3 | <1 pM | <1 pM | <1 pM |

| | Generation | Generation | Generation |
|---|---|---|---|
| Clone 2-3.15 | 15 | 57 | 83 |
| Averaged peak IVCD (cell/ml) | 2.32E+07 | 2.89E+07 | 2.12E+07 |
| Averaged titer at day 13 (g/l) | 4.08 | 3.2 | 3.306 |
| Averaged IVCD (cell/ml) | 1.65E+08 | 1.63E+08 | 1.38E+08 |
| Averaged qP (pg/cell/day) | 24.88 | 22.46 | 23.23 |
| Averaged viability at day 13 (%) | 79.15 | 48.9 | 46.8 |
| Averaged aggregation (%) | 0.92% | 1.06% | 1.06% |
| Averaged Kd to human HER3 | <1 pM | <1 pM | <1 pM |

Overall, clone 2-3.12 showed the best performance in terms of productivity, long-term stability and monomeric purity.

In particular, clone 2-3.12 displayed only an 8% reduction in antibody titer between generations 19 and 88, and antibody preparations derived from culture of clone 2-3.12 across different generations displayed very low aggregation propensity and maintained sub-picomolar affinity for human HER3.

### 3.3 Bioreactor Culture

Growth and productivity of clone 2-3.12 was characterized in culture in a SmartGlass bioreactor (Finesse).

Cells of clone 2-3.12 were cultured at a concentration of 3×10⁵˜ cells/L in 30 ml of medium comprising EX-CELL Advanced CHO Fed-Batch Medium+6 mM L-Glutamine+250 nM MTX in an E1125 shake flask, and maintained at 37° C., 80% relative humidity, 8% CO₂ and 125 rpm agitation for 3-4 days.

The cells were then transferred to an E250 flask and topped up with the cell culture medium to a total volume of 60 ml. The cells were cultured for 1 day, in order to obtain an expected 6×10⁸ cells for inoculation of 2 L of cell culture medium in a 3L SmartGlass bioreactor.

The following process parameters were used for subsequent culture in the SmartGlass bioreactor:

| Parameter | Set point |
|---|---|
| Air Overlay | Constant speed 50 ml/min |
| DO % (Air Saturation) | 40% |
| pH* | 7.0 |
| Temperature | 37° C. |
| Temperature shift on Day 8 | 35° C. (VCD: 32.5E6 cell/ml) |
| Starting Volume | 2 L |
| Total Gassing | Maximum 0.2 vvm |
| Agitation (RPM) | 200 |
| Impeller diameter (mm) | 58 |
| Tip speed (m/s) | 0.6 |
| Length of cultivation | 15 days |

*Recommend setting deadband at 0.05

| PID Tunning | Set point |
|---|---|
| DO % P (Gain) | 1-1.5 |
| DO % I (Reset) | 120-180 s |
| DO % D (Rate) | 0-10 s |
| pH P (Gain) | 1 |
| pH I (Reset) | 150 s |
| pH D (Rate) | 0 |
| Temperature P (Gain) | 0.5 |
| Temperature I (Reset) | 10 s |
| Temperature D (Rate) | 0 |

The culture was fed with Cell Boost 7a (GE Healthcare, Cat #SH31 027.01) and Cell Boost 7b (GE Healthcare, Cat #SH31026.07) as follows:

| | | Cell | Cell | Feeding | Feeding |
|---|---|---|---|---|---|
| | Volume | Boost7a | Boost | speed | speed |
| | (ml) | (ml) | 7b (ml) | for 7a | for 7b |
| Day 0 | 2000 | | | | |
| Day 3 | 2000 | 80.0 | 8.0 | 7 RPM | 3.5 RPM |
| Day 5 | 2088 | 83.5 | 8.4 | 7 RPM | 3.5 RPM |
| Day 7 | 2180 | 87.2 | 8.7 | 7 RPM | 3.5 RPM |
| Day 9 | 2276 | 91.0 | 9.1 | 7 RPM | 3.5 RPM |
| Day 11 | 2376 | 95.0 | 9.5 | 7 RPM | 3.5 RPM |
| Day 15 | 2480 | | | | |
| Total Cell Boost 7a | | 437 | | | |
| 10% extra Cell | | 480 | | | |
| Boost 7a | | | | | |
| Total Cell Boost 7b | | | 44 | | |
| 10% extra Cell | | | 48 | | |
| Boost 7b | | | | | |

Sampling volume at approximately 6 ml per day and glucose feeding volume are not considered

The culture was also fed with D-(+)-glucose from day 8 onwards, topping up to a calculated concentration of 6 g/L.

The amount of glucose required to achieve a concentration of 6 g/L on a media feeding day was calculated as follows: ml of glucose needed: [(6−current glucose level in g/L−2)/450]×current volume in ml. The amount of glucose required to achieve a concentration of 6 g/L on a non-media feeding day was calculated as follows: ml of glucose needed: [(6−current glucose level in g/L)/450]×current volume in ml.

The percentage of viable cells and viable cell densities (VCD) were determined by analysis using a Vi-Cell (Beckman Coulter), and using the trypan blue exclusion method. The parameters used were as follows:

| Cell Type | CHO |
|---|---|
| Minimum diameter (microns) | 6 |
| Maximum diameter (microns) | 50 |
| number of images | 50 |
| Aspirate cycles | 1 |
| Trypan blue mixing cycles | 3 |
| Cell brightness (%) | 85 |
| Cell sharpness | 100 |
| Viable cell spot brightness (%) | 75 |
| Viable cell spot are (%) | 5 |
| Minimum circularity | 0 |
| Decluster degree | Medium |
| Instrument calibration | Focus control and |
| | concentration control |

Monoclonal antibody titer and qP were determined daily from day 3 to day 15 of bioreactor culture, as described in section 3.1 above.

The viable cell density over time was used to determine the growth rate of the cells in culture, and to calculate the cell doubling time (using the formula: doubling time=In(2)/k, where ‘k’ is the rate constant). ‘Tau’ is the time constant, and is the reciprocal of K. ‘Y0’ is the viable cell density at time=0.

The results are shown in FIGS. 3A and 3B.

The mAb titer at day 15 was calculated to be 4.6 g/L. The highest rate of monoclonal antibody production was observed from days 8 to 10, and the doubling time of the cells in culture was calculated to be 22.15 hours.

### 3.4 Conclusion

Clone 2-3.12 was identified to express 10D1F hIgG1 with high productivity (>4 g/L), with high phenotypic stability, across at least 88 generations.

Clone 2-3.12 was also found to be a fast-growing cell line (doubling time <24 hours), which is able to grow to high cell density in culture (peak cell density >40×10⁶ cells/ml).

Clone 2-3.12 is moreover able to utilise lactate produced as a by-product in culture via a lactate consumption metabolic pathway, as a result of which lactate does not accumulate.

Clone 2-3.12 was deposited 7 May 2021 as ATCC Patent Deposit Number PTA-127062.

1. A cell of the cell line deposited 7 May 2021 as ATCC Patent Deposit Number PTA-127062.

2. A population of cells of the cell line deposited 7 May 2021 as ATCC Patent Deposit Number PTA-127062.

3. A composition comprising a cell according to claim 1, or a population of cells according to claim 2.

4. A method of producing an antigen-binding molecule, comprising culturing a cell according to claim 1, or a population of cells according to claim 2, under conditions suitable for expression of the antigen-binding molecule.

5. The method according to claim 4, wherein the method comprises:
culturing a cell according to claim 1, or a population of cells according to claim 2, under conditions suitable for expression of the antigen-binding molecule; and
isolating or purifying antigen-binding molecule produced at the preceding step.

6. A method of producing a pharmaceutical composition, comprising:
culturing a cell according to claim 1, or a population of cells according to claim 2, under conditions suitable for expression of the antigen-binding molecule; and
formulating the antigen-binding molecule produced at the preceding step with a pharmaceutically-acceptable carrier, diluent, excipient or adjuvant.

7. The method according to claim 6, wherein the method comprises:
culturing a cell according to claim 1, or a population of cells according to claim 2, under conditions suitable for expression of the antigen-binding molecule;
isolating or purifying antigen-binding molecule produced at the preceding step;
formulating the isolated or purified antigen-binding molecule with a pharmaceutically-acceptable carrier, diluent, excipient or adjuvant.

8. Use of a cell according to claim 1, or a population of cells according to claim 2, in the production of an antigen-binding molecule which binds specifically to HER3.

9. Use of a cell according to claim 1, or a population of cells according to claim 2, in the production of pharmaceutical composition comprising an antigen-binding molecule which binds specifically to HER3.

10. An antigen-binding molecule, or a plurality of antigen-binding molecules, obtained by a method according to claim 4 or claim 5.

11. A pharmaceutical composition obtained by a method according to claim 6 or claim 7.

12. An antigen-binding molecule or a plurality of antigen-binding molecules according to claim 10, or a pharmaceutical composition according to claim 11, for use in a method of medical treatment or prophylaxis.

13. An antigen-binding molecule or a plurality of antigen-binding molecules according to claim 10, or a pharmaceutical composition according to claim 11, for use in a method of treating or preventing a cancer.

14. Use of an antigen-binding molecule or a plurality of antigen-binding molecules according to claim 10, or a pharmaceutical composition according to claim 11, in the manufacture of a medicament for use in a method of treating or preventing a cancer.

15. A method of treating or preventing a cancer, comprising administering to a subject a therapeutically- or prophylactically-effective amount of an antigen-binding molecule or a plurality of antigen-binding molecules according to claim 10, or a pharmaceutical composition according to claim 11.

16. The antigen-binding molecule, the plurality of antigen-binding molecules or the pharmaceutical composition for use according to claim 13, the use according to claim 14, or the method according to claim 15, wherein the cancer is selected from: a cancer comprising cells expressing an EGFR family member, a cancer comprising cells expressing HER3, a cancer comprising cells having a mutation resulting in increased expression of a ligand for HER3, a cancer comprising cells having an NRG gene fusion, a solid tumor, breast cancer, breast carcinoma, breast invasive carcinoma, ductal carcinoma, gastric cancer, gastric carcinoma, gastric adenocarcinoma, colorectal cancer, colorectal carcinoma, colorectal adenocarcinoma, head and neck cancer, squamous cell carcinoma of the head and neck (SCCHN), lung cancer, non-small cell lung cancer, lung adenocarcinoma, invasive mucinous lung adenocarcinoma, squamous cell lung carcinoma, ovarian cancer, ovarian carcinoma, ovarian serous adenocarcinoma, ovarian serous cystadenocarcinoma, renal cancer, renal cell carcinoma, renal clear cell carcinoma, renal cell adenocarcinoma, renal papillary cell carcinoma, pancreatic cancer, pancreatic adenocarcinoma, pancreatic ductal adenocarcinoma, cervical cancer, cervical squamous cell carcinoma, skin cancer, melanoma, esophageal cancer, esophageal adenocarcinoma, liver cancer, hepatocellular carcinoma, cholangiocarcinoma, gallbladder cancer, uterine cancer, endometrial cancer, uterine corpus endometrial carcinoma, uterine carcinosarcoma, thyroid cancer, thyroid carcinoma, pheochromocytoma, paraganglioma, bladder cancer, bladder urothelial carcinoma, prostate cancer, prostate adenocarcinoma, sarcoma, soft tissue sarcoma, thymoma, neuroendocrine tumor and neuroendocrine tumor of the nasopharynx.

17. A method of inhibiting HER3-mediated signalling, comprising contacting HER3-expressing cells with an antigen-binding molecule or a plurality of antigen-binding molecules according to claim 10, or a pharmaceutical composition according to claim 11.

18. A method of reducing the number or activity of HER3-expressing cells, the method comprising contacting HER3-expressing cells with an antigen-binding molecule or a plurality of antigen-binding molecules according to claim 10, or a pharmaceutical composition according to claim 11.

19. An in vitro complex, optionally isolated, comprising an antigen-binding molecule according to claim 10 bound to HER3.

20. A method for detecting HER3 in a sample, comprising contacting a sample containing, or suspected to contain, HER3 with an antigen-binding molecule or a plurality of antigen-binding molecules according to claim 10, or a pharmaceutical composition according to claim 11, and detecting the formation of a complex of the antigen-binding molecule with HER3.

21. A method of selecting or stratifying a subject for treatment with a HER3-targeted agent, the method comprising contacting, in vitro, a sample from the subject with an antigen-binding molecule or a plurality of antigen-binding molecules according to claim 10, or a pharmaceutical composition according to claim 11, and detecting the formation of a complex of the antigen-binding molecule with HER3.

22. Use of an antigen-binding molecule or a plurality of antigen-binding molecules according to claim 10, or a pharmaceutical composition according to claim 11, as an in vitro or in vivo diagnostic or prognostic agent.

23. Use of an antigen-binding molecule or a plurality of antigen-binding molecules according to claim 10, or a pharmaceutical composition according to claim 11, in a method for detecting, localizing or imaging a cancer, optionally wherein the cancer is selected from: a cancer comprising cells expressing an EGFR family member, a cancer comprising cells expressing HER3, a cancer comprising cells having a mutation resulting in increased expression of a ligand for HER3, a cancer comprising cells having an NRG gene fusion, a solid tumor, breast cancer, breast carcinoma, breast invasive carcinoma, ductal carcinoma, gastric cancer, gastric carcinoma, gastric adenocarcinoma, colorectal cancer, colorectal carcinoma, colorectal adenocarcinoma, head and neck cancer, squamous cell carcinoma of the head and neck (SCCHN), lung cancer, non-small cell lung cancer, lung adenocarcinoma, invasive mucinous lung adenocarcinoma, squamous cell lung carcinoma, ovarian cancer, ovarian carcinoma, ovarian serous adenocarcinoma, ovarian serous cystadenocarcinoma, renal cancer, renal cell carcinoma, renal clear cell carcinoma, renal cell adenocarcinoma, renal papillary cell carcinoma, pancreatic cancer, pancreatic adenocarcinoma, pancreatic ductal adenocarcinoma, cervical cancer, cervical squamous cell carcinoma, skin cancer, melanoma, esophageal cancer, esophageal adenocarcinoma, liver cancer, hepatocellular carcinoma, cholangiocarcinoma, gallbladder cancer, uterine cancer, endometrial cancer, uterine corpus endometrial carcinoma, uterine carcinosarcoma, thyroid cancer, thyroid carcinoma, pheochromocytoma, paraganglioma, bladder cancer, bladder urothelial carcinoma, prostate cancer, prostate adenocarcinoma, sarcoma, soft tissue sarcoma, thymoma, neuroendocrine tumor and neuroendocrine tumor of the nasopharynx.

24. A kit of parts, comprising: a cell according to claim 1, a population of cells according to claim 2, a composition according to claim 3, an antigen-binding molecule or a plurality of antigen-binding molecules according to claim 10, or a pharmaceutical composition according to claim 11.
